(19) United States
(12) Patent Application Publication
(10) Pub. No.: US 2024/0398704 A1
(43) Pub. Date: Dec. 5, 2024

(54) ORAL LIQUID ENZALUTAMIDE COMPOSITIONS

(71) Applicant: FERRING B.V., Hoofddorp (NL)

(72) Inventors: BijayKumar PADHI, Hyderabad (IN); Shailesh Vishwanath BIRADAR, Hyderabad (IN); Ambedkar Sunil SONGA, Hyderabad (IN)

(73) Assignee: FERRING B.V., Hoofddorp (NL)

(21) Appl. No.: 18/697,370

(22) PCT Filed: Sep. 30, 2022
(86) PCT No.: PCT/IB2022/059345

§ 371 Date: Mar. 29, 2024

(30) Foreign Application Priority Data
Oct. 1, 2021 (IN) 202111044703

(51) Int. Cl. A61K 9/00; A61K 9/107; A61K 31/4166; A61K 47/10; A61K 47/14; A61K 47/44

(52) U.S. Cl. CPC A61K 9/0095; A61K 9/107; A61K 31/4166; A61K 47/10; A61K 47/14; A61K 47/44

(57) ABSTRACT

Described herein are oral liquid pharmaceutical compositions comprising enzalutamide and therapeutic methods for using them.

### CROSS-REFERENCE TO RELATED APPLICATIONS

This application claims priority to Indian patent application No. 202111044703, filed Oct. 1, 2021, the entire contents of which are incorporated herein by reference.

### FIELD

Described herein are oral liquid pharmaceutical compositions comprising enzalutamide and therapeutic uses thereof.

### BACKGROUND

Enzalutamide is a small molecule androgen receptor inhibitor. Enzalutamide has been shown to competitively inhibit androgen binding to androgen receptors, and consequently, inhibits nuclear translocation of androgen receptors and their interaction with DNA. Enzalutamide has been indicated for use in the treatment of patients with castration-resistant prostate cancer and metastatic castration-sensitive prostate cancer.

Oral solid compositions of enzalutamide have been approved for use. XTANDI® is available in film-coated tablet and liquid-filled soft gelatin capsule dosage forms. The tablets are available in 40 mg and 80 mg doses. Each capsule contains 40 mg of enzalutamide as a solution in caprylocaproyl polyoxylglycerides, with, butylated hydroxyanisole, butylated hydroxytoluene, gelatin, sorbitol sorbitan solution, glycerin, purified water, titanium dioxide, polyvinyl acetate and black iron oxide as the inactive ingredients. The recommend dose of XTANDI® is 160 mg once daily. Thus, patients must take four 40 mg capsules, four 40 mg tablets, or two 80 mg tablets daily, as a single event/time dose.

Oral liquid compositions of enzalutamide have been disclosed in WO 2018/037310. However, the disclosed formulations include large amounts of LABRASOL® and propylene glycol. LABRASOL® is reported to be bitter (see, e.g., Isabelle et. al, Advanced Drug Delivery Reviews 142:128-140 (2019), thus the compositions of WO 2018/037310 are not palatable.

Thus, there remains a need for enzalutamide pharmaceutical compositions, and for oral liquid enzalutamide compositions in particular.

### SUMMARY

Provided herein are oral liquid pharmaceutical enzalutamide compositions comprising (i) enzalutamide; (ii) optionally, an oil; (iii) a surfactant and/or a solubilizer, (iv) a co-surfactant, (v) an antioxidant, (vi) optionally, a precipitation inhibitor; and (vii) optionally, water.

Provided in one aspect are oral liquid pharmaceutical compositions of enzalutamide, comprising:
(i) about 2.5% to about 6% w/w of enzalutamide;
(ii) optionally, about 0.5% to about 25% w/w of an oil selected from one or more of a vegetable oil, a flavored oil, and an essential oil;
(iii) about 5% to about 85% w/w of one or both of a surfactant and a solubilizer, wherein the surfactant comprises one or more selected from tocopherol polyethylene glycol succinate surfactants, polyoxyethylene castor oil surfactants, polyoxyl hydrogenated castor oil surfactants, polyoxyl hydroxy stearate surfactants, lauroyl polyoxylglyceride and lauroyl macrogoglyceride surfactants, stearoyl polyoxylglyceride and stearoyl macrogoglyceride surfactants, and polyoxyl stearate surfactants, and the solubilizer comprises one or more selected from polyethylene glycol caprylic/capric glyceride solubilizers, polyglyceryl oleate solubilizers, and polyoxyethylene sorbitan monooleate solubilizers;
(iv) about 5% to about 25% w/w of a co-surfactant selected from one or more of propylene glycol monolaurate Type II surfactants, oleoyl polyoxyl-6-glyceride surfactants, linoleoyl macrogolglyceride and linoleoyl polyoxylglyceride surfactants, lauroyl macrogolglyceride and lauroyl polyoxylglyceride surfactants, glycerol monocaprylocaprate Type I surfactants, and propylene glycol monocaprylate Type II surfactants;
(v) about 0.01% to about 1% w/w of an antioxidant comprising one or more selected from DL-methionine, butylated hydroxy anisole (BHA), butylated hydroxy toluene (BHT), arginine, cysteine, ascorbic palmitate, sodium metabisulfite, sodium thiosulfate, propyl gallate, gamma linolenic acid, ethylenediaminetetraacetic acid (EDTA), ascorbic acid, and alpha-tocopherol;
(vi) optionally, about 0.5% to about 5% w/w of precipitation inhibitor selected one or more of polyvinyl caprolactam-polyvinyl acetate-polyethylene glycol graft copolymer solubilizers, polyoxyethylene polyoxypropylene glycol solubilizers, polyvinylpyrrolidone solubilizers, vinylpyrrolidone-vinyl acetate copolymer solubilizers, polyvinyl alcohol/polyethylene glycol graft copolymer solubilizers, hydroxypropyl methylcellulose solubilizers, and hypromellose acetate succinate solubilizers; and
(vii) optionally, water (such as about 1% to about 5% w/w water).

Provided in another aspect are oral liquid pharmaceutical compositions of enzalutamide, comprising:
(i) about 2.5% to about 6% w/w of enzalutamide;
(ii) optionally, about 0.5% to about 25% w/w of an oil selected from one or more of a vegetable oil, a flavored oil, and an essential oil;
(iii) about 5% to about 65% w/w of a surfactant, wherein the surfactant comprises one or more selected from tocopherol polyethylene glycol succinate surfactants, polyoxyethylene castor oil surfactants, polyoxyl hydrogenated castor oil surfactants, polyoxyl hydroxy stearate surfactants, lauroyl polyoxylglyceride and lauroyl macrogoglyceride surfactants, stearoyl polyoxylglyceride and stearoyl macrogoglyceride surfactants, and polyoxyl stearate surfactants;
(iv) about 5% to about 30% w/w of a co-surfactant selected from one or more of propylene glycol monolaurate Type II surfactants, oleoyl polyoxyl-6-glyceride surfactants, linoleoyl macrogolglyceride and linoleoyl polyoxylglyceride surfactants, lauroyl macrogolglyceride and lauroyl polyoxylglyceride surfactants, glycerol monocaprylocaprate Type I surfactants, and propylene glycol monocaprylate Type II surfactants;
(v) about 0.01% to about 1% w/w of an antioxidant comprising one or more selected from DL-methionine, butylated hydroxy anisole (BHA), butylated hydroxy toluene (BHT), arginine, cysteine, ascorbic palmitate, sodium metabisulfite, sodium thiosulfate, propyl gallate, gamma linolenic acid, ethylenediaminetetraacetic acid (EDTA), ascorbic acid, and alpha-tocopherol;
(vi) optionally, about 0.5% to about 5% w/w of precipitation inhibitor selected one or more of polyvinyl caprolactam-polyvinyl acetate-polyethylene glycol graft copolymer solubilizers, polyoxyethylene polyoxypropylene glycol solubilizers, polyvinylpyrrolidone solubilizers, vinylpyrrolidone-vinyl acetate copolymer solubilizers, polyvinyl alcohol/polyethylene glycol graft copolymer solubilizers, hydroxypropyl methylcellulose solubilizers, and hypromellose acetate succinate solubilizers; and
(vii) optionally, water (such as about 1% to about 5% w/w water).

In some embodiments, the composition comprises about 20 to about 45 mg of enzalutamide per 1 mL of the composition. In some embodiments, the composition comprises about 160 mg of enzalutamide per 2.5 to 6.0 mL of the composition. In some embodiments, the composition comprises about 160 mg of enzalutamide per 6.0 mL of the composition. In some embodiments, the composition comprises 160 mg of enzalutamide per 6.0 mL of the composition. In some embodiments, the composition is palatable.

In some embodiments, the composition comprises the oil. When present, the composition may comprise about 0.5% to about 20% w/w of the oil, optionally about 0.5% to about 5% w/w of the flavored oil or essential oil and about 3% to about 15% w/w of the vegetable oil. In some embodiments, the composition comprises a flavored oil selected from one or more of peppermint oil, liquorice oil, butterscotch oil, and aniseed oil. In some embodiments, the composition comprises a vegetable oil selected from one or more of corn oil, linseed oil, and rapeseed oil.

In some embodiments, the composition comprises the surfactant. In some embodiments, the composition comprises about 5% to about 65% w/w of the surfactant, including about 5% to about 55% w/w of the surfactant, e.g., wherein the surfactant comprises one or more selected from tocopherol polyethylene glycol succinate surfactants, polyoxyethylene castor oil surfactants, polyoxyl hydrogenated castor oil surfactants, polyoxyl hydroxy stearate surfactants, lauroyl polyoxylglyceride and lauroyl macrogoglyceride surfactants, stearoyl polyoxylglyceride and stearoyl macrogoglyceride surfactants, and polyoxyl stearate surfactants. In any embodiments, the composition may comprise about 5% to about 50% w/w of the surfactant.

In some embodiments, the surfactant comprises a tocopherol polyethylene glycol succinate surfactant and a polyoxyethylene castor oil surfactant, optionally wherein the tocopherol polyethylene glycol succinate surfactant is vitamin E TPGS and/or the polyoxyethylene castor oil surfactant is PEG 35 castor oil.

In some embodiments, the composition comprises or further comprises the solubilizer. In some embodiments, the composition comprises about 10% to about 65% w/w of the solubilizer, e.g., wherein the solubilizer comprises one or more selected from polyethylene glycol caprylic/capric glyceride solubilizers, polyglyceryl oleate solubilizers, and polyoxyethylene sorbitan monooleate solubilizers. In some embodiments, the solubilizer comprises a polyethylene glycol caprylic/capric glycerides solubilizer. In some embodiments, the solubilizer is ACCONON® MC8-2.

In any embodiments, the composition may comprise about 5% to about 30% w/w or about 5% to about 25% w/w or about 5% to about 20% w/w of the co-surfactant. In some embodiments, the co-surfactant comprises a propylene glycol monolaurate Type II surfactant, oleoyl polyoxyl-6-glyceride surfactant, glycerol monocaprylocaprate Type I surfactant, or propylene glycol monocaprylate Type II surfactant, optionally wherein the propylene glycol monolaurate Type II surfactant is CAPMUL® PG-12, the oleoyl polyoxyl-6-glyceride surfactant is LABRAFIL® M 1944 CS, the glycerol monocaprylocaprate Type I surfactant is CAPMUL® MCM, and/or the propylene glycol monocaprylate Type II surfactant is CAPMUL® PG-8.

In some embodiments, the antioxidant comprises one or more selected from DL-methionine, butylated hydroxy anisole (BHA), butylated hydroxy toluene (BHT), arginine, cysteine, ascorbic palmitate, sodium metabisulfite, sodium thiosulfate, propyl gallate, gamma linolenic acid, ethylenediaminetetraacetic acid (EDTA), ascorbic acid, and alpha-tocopherol. In some embodiments, the antioxidant comprises one or more selected from DL-methionine, BHA, and BHT. In some embodiments, the antioxidant comprises DL-methionine. In some embodiments, the composition comprises about 0.05% to about 1% w/w of an antioxidant.

In some embodiments, the composition comprises the precipitation inhibitor. When present the composition may comprise about 0.5% to about 1% w/w of the precipitation inhibitor. The precipitation inhibitor may comprise a polyvinyl caprolactam-polyvinyl acetate-polyethylene glycol graft copolymer solubilizer, optionally wherein the precipitation inhibitor is SOLUPLUS®. In some embodiments, composition comprises the oil and the precipitation inhibitor.

In some embodiments, the composition further comprises about 10% to about 35% w/w of a co-solvent, wherein the co-solvent comprises one or more selected from diethylene glycol monoethyl ether, glycofurol, and N-methyl pyrrolidone. In some embodiments, the co-solvent is a diethylene glycol monoethyl ether co-solvent.

In some embodiments, the composition further comprises one or more of a sweetening agent and a flavoring agent. In some embodiments, the sweetening agent comprises one or more selected from sucralose, ammonium glycyrrhizinate, saccharin, aspartame, acesulfame potassium, cyclamate, erythritol, and neotame, and the flavoring agent comprises one or more selected from vanilla flavor, banana flavor, grapefruit flavor, strawberry flavor, raspberry flavor, bubble gum flavor, and caramel flavor. In some embodiments, the sweetening agent comprises one or more selected from sucralose and ammonium glycyrrhizinate, and the flavoring agent comprises one or more selected from vanilla flavor and banana flavor.

In some embodiments, the composition comprises peppermint oil as a flavored oil and corn oil as a vegetable oil; the surfactant is present and is a tocopherol polyethylene glycol succinate surfactant; the co-surfactant is a propylene glycol monolaurate Type II co-surfactant, the solubilizer is present and is a polyethylene glycol caprylic/capric glyceride solubilizer, the precipitation inhibitor is present and is a polyvinyl caprolactam-polyvinyl acetate-polyethylene glycol graft copolymer precipitation inhibitor, and water is present.

In some embodiments, the composition comprises peppermint oil as a flavored oil and corn oil as a vegetable oil; the surfactant is a tocopherol polyethylene glycol succinate surfactant or a combination of a tocopherol polyethylene glycol succinate surfactant and a polyoxyethylene castor oil surfactant, optionally, wherein the surfactant is vitamin E TPGS or vitamin E TPGS and PEG 35 castor oil; the co-surfactant is a propylene glycol monolaurate Type II co-surfactant, oleoyl polyoxyl-6-glyceride co-surfactant, a glycerol monocaprylocaprate Type I co-surfactant, or a propylene glycol monocaprylate Type II co-surfactant; and the precipitation inhibitor is present and is a polyvinyl caprolactam-polyvinyl acetate-polyethylene glycol graft copolymer precipitation inhibitor.

In some embodiments, the composition comprises:
(i) about 2.5% w/w of enzalutamide;
(ii) about 5% w/w of a flavored oil;
(iii) about 10% w/w of a vegetable oil;
(iv) about 7% w/w of the propylene glycol monolaurate Type II co-surfactant;
(v) about 15% w/w of the tocopherol polyethylene glycol succinate surfactant;
(vi) about 60% w/w of the polyethylene glycol caprylic/capric glyceride solubilizer;
(vii) about 0.5% w/w of the polyvinyl caprolactam-polyvinyl acetate-polyethylene glycol graft copolymer precipitation inhibitor;
(viii) an antioxidant;
(ix) optionally, a sweetening agent;
(x) optionally, a flavoring agent; and
(xi) water in an amount of about 1.5% w/w.

In specific embodiments of the foregoing, the flavored oil is peppermint oil; the vegetable oil is corn oil; the propylene glycol monolaurate Type II co-surfactant is CAPMUL® PG-12; the tocopherol polyethylene glycol succinate surfactant is D-α-tocopherol polyethylene glycol 1000 succinate surfactant; the polyethylene glycol caprylic/capric glycerides solubilizer is ACCONON® MC8-2; the polyvinyl caprolactam-polyvinyl acetate-polyethylene glycol graft copolymer precipitation inhibitor is SOLUPLUS®; and the antioxidant comprises one or more selected from DL methionine, BHA, and BHT. In further specific embodiments, the sweetening agent is present and comprises one or more selected from sucralose and ammonium glycyrrhizinate; and the flavoring agent is present and comprises vanilla and banana.

In some embodiments, the composition comprises: (i) about 2.5% w/w of enzalutamide;
(i) about 2.5% w/w of enzalutamide;
(ii) about 5% w/w of a flavored oil;
(iii) about 10% w/w of a vegetable oil;
(iv) about 7% w/w of the propylene glycol monolaurate Type II co-surfactant;
(v) about 15% w/w of the tocopherol polyethylene glycol succinate surfactant;
(vi) about 60% w/w of the polyethylene glycol caprylic/capric glyceride solubilizer;
(vii) about 0.5% w/w of the polyvinyl caprolactam-polyvinyl acetate-polyethylene glycol graft copolymer precipitation inhibitor;
(viii) an antioxidant;
(ix) optionally, a sweetening agent;
(x) optionally, a flavoring agent; and
(xi) water in an amount of about 3% w/w.

In specific embodiments of the foregoing, the flavored oil is peppermint oil; the vegetable oil is corn oil; the propylene glycol monolaurate Type II co-surfactant is CAPMUL® PG-12; the tocopherol polyethylene glycol succinate surfactant is D-α-tocopherol polyethylene glycol 1000 succinate surfactant; the polyethylene glycol caprylic/capric glycerides solubilizer is ACCONON® MC8-2; the polyvinyl caprolactam-polyvinyl acetate-polyethylene glycol graft copolymer precipitation inhibitor is SOLUPLUS®; and the antioxidant comprises DL methionine. In further specific embodiments, the sweetening agent is present and comprises one or more selected from sucralose and ammonium glycyrrhizinate; and the flavoring agent is present and comprises vanilla and banana.

In some embodiments, the composition comprises:
(i) about 2.5% w/w of enzalutamide;
(ii) about 5% w/w of a flavored oil;
(iii) about 10% w/w of a vegetable oil;
(iv) about 7% w/w of the propylene glycol monolaurate Type II co-surfactant;
(v) about 15% w/w of the tocopherol polyethylene glycol succinate surfactant;
(vi) about 60% w/w of the polyethylene glycol caprylic/capric glyceride solubilizer;
(vii) about 0.5% w/w of the polyvinyl caprolactam-polyvinyl acetate-polyethylene glycol graft copolymer precipitation inhibitor;
(viii) an antioxidant;
(ix) optionally, a sweetening agent;
(x) optionally, a flavoring agent; and
(xi) water in an amount of about 1.5% w/w.

In specific embodiments of the foregoing, the flavored oil is peppermint oil; the vegetable oil is corn oil; the propylene glycol monolaurate Type II co-surfactant is CAPMUL® PG-12; the tocopherol polyethylene glycol succinate surfactant is D-α-tocopherol polyethylene glycol 1000 succinate surfactant; the polyethylene glycol caprylic/capric glycerides solubilizer is ACCONON® MC8-2; the polyvinyl caprolactam-polyvinyl acetate-polyethylene glycol graft copolymer precipitation inhibitor is SOLUPLUS®; and the antioxidant comprises DL methionine. In further specific embodiments, the sweetening agent is present and comprises one or more selected from sucralose and ammonium glycyrrhizinate; and the flavoring agent is present and comprises vanilla and banana.

In some embodiments, the composition comprises:
(i) about 2.5% w/w of enzalutamide;
(ii) about 5% w/w of a flavored oil;
(iii) about 10% w/w of a vegetable oil;
(iv) about 7% w/w of the propylene glycol monolaurate Type II co-surfactant;
(v) about 15% w/w of the tocopherol polyethylene glycol succinate surfactant;
(vi) about 60% w/w of the polyethylene glycol caprylic/capric glyceride solubilizer;
(vii) an antioxidant;
(viii) optionally, a sweetening agent;
(ix) optionally, a flavoring agent; and
(x) water in an amount of about 3% w/w.

In specific embodiments of the foregoing, the flavored oil is peppermint oil; the vegetable oil is corn oil; the propylene glycol monolaurate Type II co-surfactant is CAPMUL® PG-12; the tocopherol polyethylene glycol succinate surfactant is D-α-tocopherol polyethylene glycol 1000 succinate surfactant; the polyethylene glycol caprylic/capric glycerides solubilizer is ACCONON® MC8-2; and the antioxidant comprises DL methionine. In further specific embodiments, the sweetening agent is present and comprises one or more selected from sucralose and ammonium glycyrrhizinate; and the flavoring agent is present and comprises vanilla and banana.

In some embodiments, the composition comprises:
(i) about 2.5% w/w of enzalutamide;
(ii) about 7% w/w of the propylene glycol monolaurate Type II co-surfactant;
(iii) about 15% w/w of the tocopherol polyethylene glycol succinate surfactant;
(iv) about 60% w/w of the polyethylene glycol caprylic/capric glyceride solubilizer;
(v) about 0.5% w/w of the polyvinyl caprolactam-polyvinyl acetate-polyethylene glycol graft copolymer precipitation inhibitor;
(vi) an antioxidant;
(vii) optionally, a sweetening agent;
(viii) optionally, a flavoring agent; and
(ix) water in an amount of about 3% w/w.

In specific embodiments of the foregoing, the propylene glycol monolaurate Type II co-surfactant is CAPMUL® PG-12; the tocopherol polyethylene glycol succinate surfactant is D-α-tocopherol polyethylene glycol 1000 succinate surfactant; the polyethylene glycol caprylic/capric glycerides solubilizer is ACCONON® MC8-2; the polyvinyl caprolactam-polyvinyl acetate-polyethylene glycol graft copolymer precipitation inhibitor is SOLUPLUS®; and the antioxidant comprises DL methionine. In further specific embodiments, the sweetening agent is present and comprises one or more selected from sucralose and ammonium glycyrrhizinate; and the flavoring agent is present and comprises vanilla and banana.

In some embodiments, the composition comprises:
(i) about 2.5% w/w of enzalutamide;
(ii) about 5% w/w of a flavored oil;
(iii) about 10% w/w of a vegetable oil;
(iv) about 7% w/w of the propylene glycol monolaurate Type II co-surfactant;
(v) about 15% w/w of the tocopherol polyethylene glycol succinate surfactant;
(vi) about 60% w/w of the polyethylene glycol caprylic/capric glyceride solubilizer;
(vii) about 0.5% w/w of the polyvinyl caprolactam-polyvinyl acetate-polyethylene glycol graft copolymer precipitation inhibitor;
(viii) an antioxidant;
(ix) optionally, a sweetening agent;
(x) optionally, a flavoring agent; and
(xi) water in an amount of about 3% w/w.

In specific embodiments of the foregoing, the flavored oil is peppermint oil; the vegetable oil is corn oil; the propylene glycol monolaurate Type II co-surfactant is CAPMUL® PG-12; the tocopherol polyethylene glycol succinate surfactant is D-α-tocopherol polyethylene glycol 1000 succinate surfactant; the polyethylene glycol caprylic/capric glycerides solubilizer is ACCONON® MC8-2; the polyvinyl caprolactam-polyvinyl acetate-polyethylene glycol graft copolymer precipitation inhibitor is SOLUPLUS®; and the antioxidant comprises arginine or cysteine. In further specific embodiments, the sweetening agent is present and comprises one or more selected from sucralose and ammonium glycyrrhizinate; and the flavoring agent is present and comprises vanilla and banana.

In some embodiments, the composition comprises:
(i) about 2.5% w/w of enzalutamide;
(ii) about 5% w/w of a flavored oil;
(iii) about 10% w/w of a vegetable oil;
(iv) about 7% w/w of the propylene glycol monolaurate Type II co-surfactant;
(v) about 15% w/w of the tocopherol polyethylene glycol succinate surfactant;
(vi) about 60% w/w of the polyethylene glycol caprylic/capric glyceride solubilizer;
(vii) about 0.5% w/w of the polyvinyl caprolactam-polyvinyl acetate-polyethylene glycol graft copolymer precipitation inhibitor;
(viii) an antioxidant;
(ix) optionally, a sweetening agent;
(x) optionally, a flavoring agent; and
(xi) water in an amount of about 1.5% w/w.

In specific embodiments of the foregoing, the flavored oil is peppermint oil; the vegetable oil is corn oil; the propylene glycol monolaurate Type II co-surfactant is CAPMUL® PG-12; the tocopherol polyethylene glycol succinate surfactant is D-α-tocopherol polyethylene glycol 1000 succinate surfactant; the polyethylene glycol caprylic/capric glycerides solubilizer is ACCONON® MC8-2; the polyvinyl caprolactam-polyvinyl acetate-polyethylene glycol graft copolymer precipitation inhibitor is SOLUPLUS®; and the antioxidant comprises DL methionine. In further specific embodiments, the sweetening agent is present and comprises one or more selected from sucralose and ammonium glycyrrhizinate; and the flavoring agent is present and comprises vanilla and banana.

In some embodiments, the composition comprises:
(i) about 3% w/w of enzalutamide;
(ii) about 1.0% w/w of a flavored oil;
(iii) about 3.5% w/w of a vegetable oil;
(iv) about 13% w/w of the oleoyl polyoxyl-6-glyceride co-surfactant;
(v) about 12% w/w of the tocopherol polyethylene glycol succinate surfactant;
(vi) about 20% w/w of the diethylene glycol monoethyl ether co-solvent;
(vii) about 26% w/w of the polyethylene glycol caprylic/capric glyceride solubilizer;
(viii) about 0.75% w/w of the polyvinyl caprolactam-polyvinyl acetate-polyethylene glycol graft copolymer precipitation inhibitor;
(ix) about 21% w/w of the polyoxyethylene castor oil surfactant,
(x) an antioxidant;
(xi) optionally, a sweetening agent; and
(xii) optionally, a flavoring agent.

In specific embodiments of the foregoing, the flavored oil is peppermint oil; the vegetable oil is corn oil; the oleoyl polyoxyl-6-glyceride co-surfactant is LABRAFIL® M 1944 CS; the tocopherol polyethylene glycol succinate surfactant is vitamin E TPGS; the diethylene glycol monoethyl ether co-solvent is TRANSCUTOL® HP; the polyethylene glycol caprylic/capric glyceride solubilizer is ACCONON® MC8-2; the polyvinyl caprolactam-polyvinyl acetate-polyethylene glycol graft copolymer precipitation inhibitor is SOLUPLUS®; the polyoxyethylene castor oil surfactant is KOLLIPHOR® ELP; the antioxidant comprises one or more selected from butylated hydroxy anisole (BHA) and butylated hydroxy toluene (BHT); the sweetening agent is present and comprises one or more selected from sucralose and ammonium glycyrrhizinate; and the flavoring agent is present and comprises vanilla.

In some embodiments, the composition comprises:
(i) about 4% w/w of enzalutamide;
(ii) about 1.5% w/w of a flavored oil;
(iii) about 5% w/w of a vegetable oil;
(iv) about 17% w/w of the oleoyl polyoxyl-6-glyceride co-surfactant;
(v) about 6% w/w of the tocopherol polyethylene glycol succinate surfactant;
(vi) about 25% w/w of the diethylene glycol monoethyl ether co-solvent;
(vii) about 40% w/w of the polyethylene glycol caprylic/capric glyceride solubilizer;
(viii) about 0.5% w/w of the polyvinyl caprolactam-polyvinyl acetate-polyethylene glycol graft copolymer precipitation inhibitor;
(ix) an antioxidant;
(x) optionally, a sweetening agent; and
(xi) optionally, a flavoring agent.

In specific embodiments of the foregoing, the flavored oil is peppermint oil; the vegetable oil is corn oil; the oleoyl polyoxyl-6-glyceride co-surfactant is LABRAFIL® M 1944 CS; the tocopherol polyethylene glycol succinate surfactant is D-α-tocopherol polyethylene glycol 1000 succinate surfactant; the diethylene glycol monoethyl ether co-solvent is TRANSCUTOL® HP; the polyethylene glycol caprylic/capric glycerides solubilizer is ACCONON® MC8-2; the polyvinyl caprolactam-polyvinyl acetate-polyethylene glycol graft copolymer precipitation inhibitor is SOLUPLUS®; the antioxidant comprises one or more selected from butylated hydroxy anisole (BHA) and butylated hydroxy toluene (BHT); the sweetening agent is present and comprises one or more selected from sucralose and ammonium glycyrrhizinate; and the flavoring agent is present and comprises vanilla.

In some embodiments, the composition comprises:
(i) about 3% w/w of enzalutamide;
(ii) about 5% w/w of a flavored oil;
(iii) about 12% w/w of a vegetable oil;
(iv) about 13% w/w of the oleoyl polyoxyl-6-glyceride co-surfactant;
(v) about 18% w/w of the tocopherol polyethylene glycol succinate surfactant;
(vi) about 20% w/w of the diethylene glycol monoethyl ether co-solvent;
(vii) about 0.75% w/w of the polyvinyl caprolactam-polyvinyl acetate-polyethylene glycol graft copolymer precipitation inhibitor;
(viii) about 30% w/w of the polyoxyethylene castor oil surfactant,
(ix) an antioxidant;
(x) optionally, a sweetening agent; and
(xi) optionally, a flavoring agent.

In specific embodiments of the foregoing, the flavored oil is peppermint oil; the vegetable oil is corn oil; the oleoyl polyoxyl-6-glyceride co-surfactant is LABRAFIL® M 1944 CS; the tocopherol polyethylene glycol succinate surfactant is vitamin E TPGS; the diethylene glycol monoethyl ether co-solvent is TRANSCUTOL® HP; the polyvinyl caprolactam-polyvinyl acetate-polyethylene glycol graft copolymer precipitation inhibitor is SOLUPLUS®; the polyoxyethylene castor oil surfactant is KOLLIPHOR® ELP; the antioxidant comprises one or more selected from butylated hydroxy anisole (BHA) and butylated hydroxy toluene (BHT); the sweetening agent is present and comprises one or more selected from sucralose and ammonium glycyrrhizinate; and the flavoring agent is present and comprises vanilla.

In some embodiments, the composition comprises:
(i) about 3% w/w of enzalutamide;
(ii) about 5% w/w of a flavored oil;
(iii) about 11% w/w of a vegetable oil;
(iv) about 7% w/w of the glycerol monocaprylocaprate Type I co-surfactant;
(v) about 17% w/w of the tocopherol polyethylene glycol succinate surfactant;
(vi) about 55% w/w of the polyethylene glycol caprylic/capric glyceride solubilizer;
(vii) about 0.5% w/w of the polyvinyl caprolactam-polyvinyl acetate-polyethylene glycol graft copolymer precipitation inhibitor;
(viii) an antioxidant;
(ix) optionally, a sweetening agent; and
(x) optionally, a flavoring agent.

In specific embodiments of the foregoing, the flavored oil is peppermint oil; the vegetable oil is corn oil; the glycerol monocaprylocaprate Type I co-surfactant is CAPMUL® MCM; the tocopherol polyethylene glycol succinate surfactant is D-α-tocopherol polyethylene glycol 1000 succinate surfactant; the polyethylene glycol caprylic/capric glycerides solubilizer is ACCONON® MC8-2; the polyvinyl caprolactam-polyvinyl acetate-polyethylene glycol graft copolymer precipitation inhibitor is SOLUPLUS®; the antioxidant comprises one or more selected from butylated hydroxy anisole (BHA) and butylated hydroxy toluene (BHT); the sweetening agent is present and comprises one or more selected from sucralose and ammonium glycyrrhizinate; and the flavoring agent is present and comprises vanilla.

In some embodiments, the composition comprises:
(i) about 3% w/w of enzalutamide;
(ii) about 5% w/w of a flavored oil;
(iii) about 11% w/w of a vegetable oil;
(iv) about 7% w/w of the propylene glycol monocaprylate Type II co-surfactant;
(v) about 17% w/w of the tocopherol polyethylene glycol succinate surfactant;
(vi) about 55% w/w of the polyethylene glycol caprylic/capric glyceride solubilizer;
(vii) about 0.5% w/w of the polyvinyl caprolactam-polyvinyl acetate-polyethylene glycol graft copolymer precipitation inhibitor;
(viii) an antioxidant;
(ix) optionally, a sweetening agent; and
(x) optionally, a flavoring agent.

In specific embodiments of the foregoing, the flavored oil is peppermint oil; the vegetable oil is corn oil; the propylene glycol monocaprylate Type II co-surfactant is CAPMUL® PG-8; the tocopherol polyethylene glycol succinate surfactant is D-α-tocopherol polyethylene glycol 1000 succinate surfactant; the polyethylene glycol caprylic/capric glycerides solubilizer is ACCONON® MC8-2; the polyvinyl caprolactam-polyvinyl acetate-polyethylene glycol graft copolymer precipitation inhibitor is SOLUPLUS®; the antioxidant comprises one or more selected from butylated hydroxy anisole (BHA) and butylated hydroxy toluene (BHT); the sweetening agent is present and comprises one or more selected from sucralose and ammonium glycyrrhizinate; and the flavoring agent is present and comprises vanilla.

In some embodiments, the composition comprises:
(i) about 3% w/w of enzalutamide;
(ii) about 5% w/w of a flavored oil;
(iii) about 11% w/w of a vegetable oil;
(iv) about 7% w/w of the propylene glycol monolaurate Type II co-surfactant;
(v) about 17% w/w of the tocopherol polyethylene glycol succinate surfactant;
(vi) about 55% w/w of the polyethylene glycol caprylic/capric glyceride solubilizer;
(vii) about 0.5% w/w of the polyvinyl caprolactam-polyvinyl acetate-polyethylene glycol graft copolymer precipitation inhibitor;
(viii) an antioxidant;
(ix) optionally, a sweetening agent; and
(x) optionally, a flavoring agent.

In specific embodiments of the foregoing, the flavored oil is peppermint oil; the vegetable oil is corn oil; the propylene glycol monolaurate Type II co-surfactant is CAPMUL® PG-12; the tocopherol polyethylene glycol succinate surfactant is D-α-tocopherol polyethylene glycol 1000 succinate surfactant; the polyethylene glycol caprylic/capric glycerides solubilizer is ACCONON® MC8-2; the polyvinyl caprolactam-polyvinyl acetate-polyethylene glycol graft copolymer precipitation inhibitor is SOLUPLUS®; the antioxidant comprises one or more selected from butylated hydroxy anisole (BHA) and butylated hydroxy toluene (BHT); the sweetening agent is present and comprises one or more selected from sucralose and ammonium glycyrrhizinate; and the flavoring agent is present and comprises vanilla.

In some embodiments, the composition comprises:
(i) about 3% w/w of enzalutamide;
(ii) about 5% w/w of a flavored oil;
(iii) about 12% w/w of a vegetable oil;
(iv) about 15% w/w of the oleoyl polyoxyl-6-glyceride co-surfactant;
(v) about 17% w/w of the tocopherol polyethylene glycol succinate surfactant;
(vi) about 19% w/w of the diethylene glycol monoethyl ether co-solvent;
(vii) about 0.75% w/w of the polyvinyl caprolactam-polyvinyl acetate-polyethylene glycol graft copolymer precipitation inhibitor;
(viii) about 28% w/w of the polyoxyethylene castor oil surfactant,
(ix) an antioxidant;
(x) optionally, a sweetening agent; and
(xi) optionally, a flavoring agent.

In specific embodiments of the foregoing, the flavored oil is peppermint oil; the vegetable oil is corn oil; the oleoyl polyoxyl-6-glyceride co-surfactant is LABRAFIL® M 1944 CS; the tocopherol polyethylene glycol succinate surfactant is vitamin E TPGS; the diethylene glycol monoethyl ether co-solvent is TRANSCUTOL® HP; the polyvinyl caprolactam-polyvinyl acetate-polyethylene glycol graft copolymer precipitation inhibitor is SOLUPLUS®; the polyoxyethylene castor oil surfactant is KOLLIPHOR® ELP; the antioxidant comprises one or more selected from butylated hydroxy anisole (BHA) and butylated hydroxy toluene (BHT); the sweetening agent is present and comprises one or more selected from sucralose and ammonium glycyrrhizinate; and the flavoring agent is present and comprises vanilla.

In some embodiments, the composition comprises:
(i) about 3% w/w of enzalutamide;
(ii) about 5% w/w of a flavored oil;
(iii) about 10% w/w of a vegetable oil;
(iv) about 7% w/w of the propylene glycol monolaurate Type II co-surfactant;
(v) about 15% w/w of the tocopherol polyethylene glycol succinate surfactant;
(vi) about 60% w/w of the polyethylene glycol caprylic/capric glyceride solubilizer;
(vii) about 0.5% w/w of the polyvinyl caprolactam-polyvinyl acetate-polyethylene glycol graft copolymer precipitation inhibitor;
(viii) an antioxidant;
(ix) optionally, a sweetening agent; and
(x) optionally, a flavoring agent.

In specific embodiments of the foregoing, the flavored oil is peppermint oil; the vegetable oil is corn oil; the propylene glycol monolaurate Type II co-surfactant is CAPMUL® PG-12; the tocopherol polyethylene glycol succinate surfactant is D-α-tocopherol polyethylene glycol 1000 succinate surfactant; the polyethylene glycol caprylic/capric glycerides solubilizer is ACCONON® MC8-2; the polyvinyl caprolactam-polyvinyl acetate-polyethylene glycol graft copolymer precipitation inhibitor is SOLUPLUS®; the antioxidant comprises one or more selected from butylated hydroxy anisole (BHA) and butylated hydroxy toluene (BHT); the sweetening agent is present and comprises one or more selected from sucralose and ammonium glycyrrhizinate; and the flavoring agent is present and comprises vanilla.

Also provided are oral liquid pharmaceutical compositions of enzalutamide, comprising:
(i) about 160 mg enzalutamide in a volume of from about 3.5 mL to about 6.0 mL;
(ii) about 0.5% to about 25% w/w of an oil selected from one or more of a vegetable oil, a flavored oil, and an essential oil;
(iii) about 5% to about 55% w/w of a surfactant, wherein the surfactant comprises one or more selected from tocopherol polyethylene glycol succinate surfactants, polyoxyethylene castor oil surfactants, polyoxyl hydrogenated castor oil surfactants, polyoxyl hydroxy stearate surfactants, lauroyl polyoxylglyceride and lauroyl macrogoglyceride surfactants, stearoyl polyoxylglyceride and stearoyl macrogoglyceride surfactants, and polyoxyl stearate surfactants;
(iv) about 5% to about 30% w/w of a co-surfactant selected from one or more of oleoyl polyoxyl-6-glyceride surfactants, linoleoyl macrogolglyceride and linoleoyl polyoxylglyceride surfactants, lauroyl macrogolglyceride and lauroyl polyoxylglyceride surfactants, glycerol monocaprylocaprate Type I surfactants, propylene glycol monocaprylate Type II surfactants, and propylene glycol monolaurate Type II surfactants; and
(v) about 0.5% to about 5% w/w of precipitation inhibitor selected one or more of polyvinyl caprolactam-polyvinyl acetate-polyethylene glycol graft copolymer solubilizers, polyoxyethylene polyoxypropylene glycol solubilizers, polyvinylpyrrolidone solubilizers, vinylpyrrolidone-vinyl acetate copolymer solubilizers, polyvinyl alcohol/polyethylene glycol graft copolymer solubilizers, hydroxypropyl methylcellulose solubilizers, and hypromellose acetate succinate solubilizers.

Also provided are oral liquid pharmaceutical compositions of enzalutamide, comprising:
(i) about 3% to about 6% w/w of enzalutamide;
(ii) about 0.5% to about 25% w/w of an oil selected from one or more of a vegetable oil, a flavored oil, and an essential oil;
(iii) about 5% to about 55% w/w of a surfactant, wherein the surfactant comprises one or more selected from tocopherol polyethylene glycol succinate surfactants, polyoxyethylene castor oil surfactants, polyoxyl hydrogenated castor oil surfactants, polyoxyl hydroxy stearate surfactants, lauroyl polyoxylglyceride and lauroyl macrogoglyceride surfactants, stearoyl polyoxylglyceride and stearoyl macrogoglyceride surfactants, and polyoxyl stearate surfactants;
(iv) about 5% to about 25% w/w of a co-surfactant selected from one or more of oleoyl polyoxyl-6-glyceride surfactants, linoleoyl macrogolglyceride and linoleoyl polyoxylglyceride surfactants, lauroyl macrogolglyceride and lauroyl polyoxylglyceride surfactants, glycerol monocaprylocaprate Type I surfactants, propylene glycol monocaprylate Type II surfactants, and propylene glycol monolaurate Type II surfactants; and
(v) about 0.5% to about 5% w/w of precipitation inhibitor selected one or more of polyvinyl caprolactam-polyvinyl acetate-polyethylene glycol graft copolymer solubilizers, polyoxyethylene polyoxypropylene glycol solubilizers, polyvinylpyrrolidone solubilizers, vinylpyrrolidone-vinyl acetate copolymer solubilizers, polyvinyl alcohol/polyethylene glycol graft copolymer solubilizers, hydroxypropyl methylcellulose solubilizers, and hypromellose acetate succinate solubilizers.

In some embodiments, the composition is packaged as a unit dose containing 160 mg enzalutamide. In some embodiments, the unit dose has a volume of from about 3.5 mL to about 6.0 mL.

In some embodiments, the composition does not include a caprylocaproyl polyoxyl-8 glyceride surfactant/solubilizer, optionally wherein the excluded caprylocaproyl polyoxyl-8 glyceride surfactant/solubilizer is one or both of LABRASOL® and LABRASOL® ALF. Embodiments excluding caprylocaproyl polyoxyl-8 glyceride surfactants/solubilizers still may (optionally) comprise polyethylene glycol caprylic/capric glyceride solubilizers (e.g., ACCONON® MC8 2, a polyethylene glycol caprylic/capric glyceride solubilizer).

In some embodiments, the composition does not include polyethylene glycol, optionally wherein the excluded polyethylene glycol is polyethylene glycol 300 (PEG 300) and/or polyethylene glycol 400 (PEG 400). In some embodiments, the composition does not include propylene glycol. In some embodiments, the composition does not include ethanol.

Also provided herein are methods of administering enzalutamide to a subject in need thereof, comprising orally administering any of the compositions described herein.

Also provided are methods of treating prostate cancer, comprising orally administering any of the compositions described herein to a subject in need thereof.

Also provided herein are oral liquid pharmaceutical compositions as described herein, for use in treating prostate cancer.

Also provided herein are uses of enzalutamide in the preparation of a medicament for treating prostate cancer, wherein the medicament comprises an oral liquid pharmaceutical composition as described herein.

### BRIEF DESCRIPTION OF THE DRAWINGS

FIG. 1 illustrates the dissolution profile of Formulations 1 and 2 described in Table 1 and XTANDI® capsules as a reference composition.

FIG. 2 illustrates the dissolution profiles of Formulations 3, 4, 5 and 6 described in Table 1 and XTANDI® capsules as a reference composition.

FIG. 3 illustrates the dissolution profiles of Formulations 7, 8 and 9 described in Table 2 and XTANDI® capsules as a reference composition.

FIG. 4 shows mean plasma concentration-time profiles after administration of Formulations 1 and 2 as described in Table 1 or XTANDI® capsules to male beagle dogs.

FIG. 5 shows mean plasma concentration-time profiles after administration of Formulations 7, 8 and 9 as described in Table 2 or XTANDI® capsules to male beagle dogs.

FIG. 6 illustrates the dissolution profile of Formulations 1, 2, 3 and 4 described in Table 1 and XTANDI® capsules as a reference composition.

FIG. 7 illustrates the dissolution profile of Formulations 5, 6, 7, 8, and 9 described in Tables 1 and 2 and XTANDI® capsules as a reference composition.

FIG. 8 illustrates the dissolution profiles of Formulations 10, 11, 12, 13, and 14 described in Table 3 and XTANDI® capsules as a reference composition.

FIG. 9 illustrates the dissolution profiles of Formulations 15, 16, and 17 described in Table 4 and XTANDI® capsules as a reference composition.

FIG. 10 illustrates the dissolution profile of Formulations 10, 11, 12, 13, and 14 described in Table 3 and XTANDI® capsules as a reference composition.

FIG. 11 illustrates the dissolution profile of Formulations 15, 16, and 17 described in Table 4 and XTANDI® capsules as a reference composition.

FIG. 12 shows mean plasma concentration-time profiles after administration of Formulation 10 or XTANDI® capsules to human subjects.

### DETAILED DESCRIPTION

The present disclosure provides oral liquid pharmaceutical compositions of enzalutamide that provide a therapeutically effective dose of enzalutamide in a convenient volume of the composition. The compositions described herein are generally palatable. Thus, the compositions described herein may promote patient compliance and adherence to treatment. Additionally, the compositions described herein reduce the pill burden of enzalutamide therapy, which may be particularly important for the target patient population which includes elderly patients who may have difficulty swallowing and patients with dysphagia. As described in more detail below, the compositions may include (i) enzalutamide; (ii) optionally, an oil selected from one or more of a vegetable oil, a flavored oil, and an essential oil; (iii) a surfactant and/or a solubilizer, (iv) a co-surfactant, (v) an antioxidant, (vi) optionally, a precipitation inhibitor, and (v) optionally, water.

### Definitions

Technical and scientific terms used herein have the meanings commonly understood by one of ordinary skill in the art to which the present invention pertains, unless otherwise defined. Any suitable materials and/or methods known to those of ordinary skill in the art can be utilized in carrying out the present invention in view of the guidance provided herein; however, specific materials and methods are described for illustrative purposes. Materials, reagents and the like to which reference is made in the following description and examples are obtainable from commercial sources, unless otherwise noted.

As used herein, the singular forms “a,” “an,” and “the” designate both the singular and the plural, unless expressly stated to designate the singular only.

As used herein, “about” when used with a numerical value means the numerical value stated as well as plus or minus 10% of the numerical value. For example, “about 10” should be understood as both “10” and “9-11.”

As used herein, a phrase in the form “A/B” or in the form “A and/or B” means (A), (B), or (A and B); a phrase in the form “at least one of A, B, and C” means (A), (B), (C), (A and B), (A and C), (B and C), or (A, B, and C).

As used herein, the terms “comprising,” “including,” and “containing” are used expansively to mean that the described compositions, methods, or kits include at least the stated elements, and may include other elements that are not specified. The phrase “consisting essentially of” is used to include those elements specifically recited and additional elements that do not materially affect the basic and novel characteristics of the claimed invention, such as ingredients that do not materially undermine the solubility of enzalutamide in the composition or the palatability of the composition.

As used herein, “subject” denotes any mammal, including humans. For example, a subject may be suffering from or at risk of developing a condition that can be treated or prevented with enzalutamide, or may be taking enzalutamide for other purposes.

The terms “administer,” “administration,” and “administering” as used herein refer to providing, giving, dosing and/or prescribing, such as by a health professional or his or her authorized agent or under his or her direction, and putting into, taking, or consuming, such as by a health professional or the subject.

The terms “treat,” “treating,” and “treatment” as used herein include alleviating, abating or ameliorating a disease or condition or one or more symptoms thereof, whether or not the disease or condition is considered to be “cured” or “healed,” and whether or not all symptoms are resolved.

As used herein, the phrases “therapeutically effective amount” and “therapeutically effective dose” refer to an amount or dose that provides the specific pharmacological effect for which the drug is administered in a subject in need of such treatment. It is emphasized that a therapeutically effective amount will not always be effective in treating the targeted condition, even though such amount or dose is deemed to be a therapeutically effective amount or dose by those of skill in the art. For convenience only, exemplary doses and therapeutically effective amounts are provided below with reference to adult human subjects. Those skilled in the art can adjust such amounts in accordance with standard practices as needed to treat a specific subject and/or condition/disease.

The disclosure of specific compounds under specific categories (e.g., “surfactant,” “solubilizer,” “co-surfactant,” “precipitation inhibitor,” etc.) is not necessarily limiting. Those skilled in the art will understand that a given compound may exhibit more than one function in a given composition, or may exhibit a different function depending on the composition in which it is used.

### Enzalutamide

The active ingredient of the compositions described herein is enzalutamide. Enzalutamide has the chemical name 4-(3-(4-cyano-3-(trifluoromethyl)phenyl)-5,5-dimethyl-4-oxo-2-thioxoimidazolidin-1-yl)-2-fluoro-N-methylbenzamide, the molecular formula C₂₁H₁₆F₄N₄O₂S, and a molecular weight of 464.44. It is registered under CAS Registry Number 915087-33-1. The structural formula of enzalutamide is set forth below:

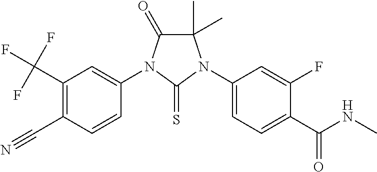 [SMILES: CNC(=O)c1ccc(N2C(=S)N(c3ccc(C#N)c(C(F)(F)F)c3)C(=O)C2(C)C)cc1F]

The compositions described herein may comprise enzalutamide in any suitable amount. For example, a composition as described herein may comprise enzalutamide in an amount of from about 2.5% to about 6% w/w, including about 2.5% w/w, about 3% w/w, about 4% w/w, about 5% w/w, about 6% w/w, and any value therebetween. A composition as described herein may comprise from about 20 mg to about 45 mg of enzalutamide per 1 mL of the composition, including from about 30 mg to about 45 mg of enzalutamide per 1 mL of the composition, including about 20 mg, about 21 mg, about 22 mg, about 23 mg, about 24, about 25 mg, about 26 mg, about 27, mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, and about 45 mg per 1 mL. In some embodiments, a composition as described herein contains about 15 mg enzalutamide per 1 mL of the composition. In some embodiments, a composition as described herein contains about 27 mg enzalutamide per 1 mL of the composition. In other embodiments, a composition as described herein contains about 30 mg enzalutamide per 1 mL of the composition. In other embodiments, a composition as described herein contains about 40 mg enzalutamide per 1 mL of the composition.

As noted above, enzalutamide typically is prescribed at a dose of about 160 mg/day. Thus, a composition as described herein may provide a therapeutically effective dose (e.g., about 160 mg) in a convenient volume of composition, such as a volume of from about 3.5 mL to about 6.0 mL, including a volume of about 5 mL and including a volume of about 6 mL. The ability to provide a therapeutically effective dose of enzalutamide in such a convenient volume of oral liquid pharmaceutical composition is advantageous for patient convenience, and may promote patient compliance with therapeutic regimens and thereby improve therapeutic outcome.

The compositions described herein may be provided in any dosage form suitable for an oral liquid pharmaceutical composition, including a bulk dosage form or unit dosage form. For a bulk dosage form, a volume of composition providing multiple doses (e.g., 150 mL or more) may be provided in a bottle. For a unit dosage form, a volume of composition providing a single dose (e.g., 3.5 to 6.0 mL) may be provided in a bottle or pouch (e.g., a stick pack or sachet). Alternatively, for a unit dosage form, a volume of composition providing a single dose or sub-dose thereof may be filled into one or more capsules. For example, a volume of composition providing ¼ of a dose (such as 1-1.25 mL or 1.5 mL) may be filled into one capsule, such that 4 capsules would provide a full dose. Likewise, a volume providing ½ of a dose (such as 2-2.5 mL or 3 mL) may be filled into one capsule, such that 2 capsules would provide a full dose. Likewise, a volume providing ⅓ of a dose (such as 1 mL of a 6 mL dose) may be filled into one capsule, such that 3 capsules would provide a full dose.

Thus, the compositions described herein may be packaged into unit dosage forms (unit doses) containing about 160 mg (including 160 mg) enzalutamide per unit dosage form (per unit dose). For example, the compositions described herein may be packaged into unit dose bottles, vials, pouches, stick packs, sachets, etc., each containing about 160 mg (including 160 mg) enzalutamide.

As illustrated in the examples below, a composition as described herein may exhibit in vitro dissolution profiles of enzalutamide comparable to that of XTANDI® capsules. Additionally or alternatively, a composition as described herein may exhibit in vivo pharmacokinetic profiles comparable to that of XTANDI® capsules, as may be reflected in mean plasma concentration-time profiles after administration. Alternatively, a composition as described herein may achieve a pharmacokinetic profile that is not comparable to that of XTANDI®, but is therapeutically effective, such as being therapeutically effective for treating prostate cancer.

As noted above, the compositions described herein typically include (i) enzalutamide; (ii) optionally, an oil; (iii) a surfactant and/or a solubilizer, (iv) a co-surfactant, (v) an antioxidant, (vi) optionally, a precipitation inhibitor, and (vii) optionally, water. In some embodiments, the compositions include (i) enzalutamide; (ii) an oil selected from one or more of a vegetable oil, a flavored oil, and an essential oil; (iii) a surfactant, (iv) a co-surfactant, (v) an antioxidant, (vi) a solubilizer, (vii) a precipitation inhibitor, and (viii) water. In some embodiments, the compositions include (i) enzalutamide; (ii) a surfactant, (iii) a co-surfactant, (iv) an antioxidant, (v) a solubilizer, (vi) a precipitation inhibitor, and (vii) water. In some embodiments, the compositions include (i) enzalutamide; (ii) an oil selected from one or more of a vegetable oil, a flavored oil, and an essential oil; (iii) a surfactant, (iv) a co-surfactant, (v) an antioxidant, and (vi) a precipitation inhibitor. In some embodiments, the compositions include (i) enzalutamide; (ii) an oil selected from one or more of a vegetable oil, a flavored oil, and an essential oil; (iii) a surfactant, (iv) a co-surfactant, (v) an antioxidant, (vi) a solubilizer, and (vii) a precipitation inhibitor. Enzalutamide having been discussed above, the other components are discussed in turn below.

### Oil

The compositions described herein optionally may comprise an oil. The oil generally acts as a vehicle or dispersant, or both, for the enzalutamide. The oil may be one or more selected from a vegetable oil, a flavored oil, and an essential oil. When present, the total oil content of the compositions described herein may be from about 0.5% to about 25% w/w or from about 0.5% to about 20% w/w.

When present the oil may comprise one or more vegetable oils. When present, the vegetable oil(s) may be present in any suitable amount. For example, a composition as described herein may comprise vegetable oil(s) in a total amount of from about 3% w/w to about 20% w/w. Thus, the total amount of vegetable oil present in a composition as described herein may be from about 3% w/w to about 20% w/w or from about 3% w/w to about 15% w/w, including about 3% w/w, about 3.5% w/w, about 4% w/w, about 4.5% w/w, about 5% w/w, about 7% w/w, about 9% w/w, about 10% w/w, about 11% w/w, about 12% w/w, about 15% w/w, about 17% w/w, or about 20% w/w, or any value therebetween.

Examples of suitable vegetable oils, include, but are not limited to, corn oil, linseed oil, and rapeseed oil. Thus, for example, a composition as described herein may comprise corn oil. For example, a composition as described herein may comprise corn oil in an amount of from about 3% w/w to about 20% w/w or from about 3% w/w to about 15% w/w, including about 3.5% w/w, about 5% w/w, about 9% w/w, about 10% w/w, about 11% w/w, and about 12% w/w.

The compositions described herein may comprise one or more of a flavored oil and an essential oil, which may act as both flavoring and vehicle (and/or dispersant) components. When present, the flavored oil and/or essential oil may be present in any suitable amount. For example, a composition as described herein may comprise a flavored oil and/or essential oil in a total amount of from about 0.5% w/w to about 5% w/w. Thus, the total amount of flavored oil and essential oil present in a composition as described herein may be about 0.5% w/w, about 1.0% w/w, about 1.5% w/w, about 2.0% w/w, about 2.5% w/w, about 3.0% w/w, about 3.5% w/w, about 4.0% w/w, about 4.5% w/w, about 5.0% w/w, or any value therebetween.

Examples of suitable flavored oils include but are not limited to, peppermint oil (which also may be considered to be an essential oil), licorice oil, butterscotch oil, and aniseed oil. Thus, for example, a composition as described herein may comprise peppermint oil. For example, a composition as described herein may comprise peppermint oil in an amount of from about 0.5% w/w to about 5% w/w, including about 1% w/w, about 1.5% w/w, about 4.0% w/w, about 4.5% w/w, or about 5.0 w/w.

Examples of suitable essential oils include but are not limited to basil (Ocimum basilicum), bergamot (Citrus bergamia), black pepper (Piper nigrum), cassia (Cinnamomum cassia), cinnamon (Cinnamomum zeylanicum), clary sage (Salvia sclarea), clove (Eugenia caryophyllata), Coriander (Coriandrum sativum), cumin (Cuminum cyminum), fennel (Foeniculum vulgare), geranium (Pelargonium graveolens), ginger (Zingiber officinale), grapefruit (Citrus X paradisi), juniper berry (Juniperus communis), lemon (Citrus limon), lemongrass (Cymbopogon flexuosus), lime (Citrus aurantifolia), marjoram (Origanum majorana), lemon balm (Melissa officinalis), oregano (Origanum vulgare), peppermint (Mentha piperita), petitgrain (Citrus aurantium), Roman chamomile (Anthemis nobilis), rosemary (Rosmarinus officinalis), spearmint (Mentha spicata), tangerine (Citrus reticulate), thyme (Thymus vulgaris), wild orange (Citrus sinensis), and Ylang (Cananga odorata).

### Surfactants

The compositions described herein may comprise one or more surfactants. Examples of suitable surfactants include those having a hydrophilic-lipophilic balance (HLB) value of from 4 to 16. Non-limiting examples of suitable surfactants include:
tocopherol polyethylene glycol succinate surfactants (e.g., D-α-tocopherol polyethylene glycol 1000 succinate, also known as Vitamin E TPGS and tocophersolan);
polyoxyethylene castor oil surfactants (e.g., polyoxyethylene 35 castor oil, also known as PEG-35 castor oil and macrogolglycerol ricinoleate, such as KOLLIPHOR® EL and KOLLIPHOR® ELP);
polyoxyl hydrogenated castor oil surfactants (e.g., polyoxyl 40 hydrogenated castor oil, also known as macrogolglycerol hydroxystearate, such as KOLLIPHOR® RH40);
polyoxyl hydroxy stearate surfactants (e.g., polyoxyl 15 hydroxy stearate, also known as macrogol (15)-hydroxystearate, polyethylene glycol (15)-hydroxystearate, and polyoxyethylated 12-hydroxystearic acid, such as KOLLIPHOR® HS 15);
lauroyl polyoxylglyceride and lauroyl macrogoglyceride surfactants (e.g., GELUCIRE® 44/14 and ACCONON® C-44);
stearoyl polyoxylglyceride and stearoyl macrogoglyceride surfactants (e.g., GELUCIRE® 50/13 and ACCONON® C-50);
polyoxyl stearate surfactants (e.g., polyoxyl stearate Type I such as GELUCIRE® 48/16);
oleoyl polyoxyl-6-glyceride surfactants (e.g., LABRAFIL® M 1944 CS);
linoleoyl macrogolglyceride and linoleoyl polyoxylglyceride surfactants (e.g., LABRAFIL® M 2125 CS);
glycerol monocaprylocaprate Type I (e.g., CAPMUL® MCM) surfactants; propylene glycol monocaprylate Type II (e.g., CAPMUL® PG-8) surfactants; and propylene glycol monolaurate Type II surfactants (e.g., CAPMUL® PG-12) and
lauroyl macrogolglyceride and lauroyl polyoxylglyceride surfactants (e.g., LABRAFIL® M 2130 CS).

Thus, for example, a composition as described herein may comprise one or more surfactants selected from tocopherol polyethylene glycol succinate surfactants (e.g., D-α-tocopherol polyethylene glycol 1000 succinate, also known as Vitamin E TPGS and tocophersolan); polyoxyethylene castor oil surfactants (e.g., polyoxyethylene 35 castor oil, also known as PEG-35 castor oil and macrogolglycerol ricinoleate, such as KOLLIPHOR® EL and KOLLIPHOR® ELP); polyoxyl hydrogenated castor oil surfactants (e.g., polyoxyl 40 hydrogenated castor oil, also known as macrogolglycerol hydroxystearate, such as KOLLIPHOR® RH40); polyoxyl hydroxy stearate surfactants (e.g., polyoxyl 15 hydroxy stearate, also known as macrogol (15)-hydroxystearate, polyethylene glycol (15)-hydroxystearate, and polyoxyethylated 12-hydroxystearic acid, such as KOLLIPHOR® HS 15); lauroyl polyoxylglyceride and lauroyl macrogoglyceride surfactants (e.g., GELUCIRER 44/14 and ACCONON® C-44); stearoyl polyoxylglyceride and stearoyl macrogoglyceride surfactants (e.g., GELUCIRE® 50/13 and ACCONON® C-50); and polyoxyl stearate surfactants (e.g., polyoxyl stearate Type I such as GELUCIRE® 48/16).

When present, the surfactant(s) can be present in a composition as described herein in any suitable amount. For example, a composition as described herein may include one or more of these surfactants in a total amount of from about 5% w/w to about 65% w/w, including about 5% w/w, about 5.5% w/w, about 6% w/w, about 6.5% w/w, about 7% w/w, about 10% w/w, about 12% w/w, about 15% w/w, about 17% w/w, about 20% w/w, about 22% w/w, about 25% w/w, about 30% w/w, about 35% w/w, about 40% w/w, about 45% w/w, about 50% w/w, about 55% w/w, about 60% w/w, or about 65% w/w, or any value therebetween. When combinations of surfactants are used, they each may be present in any suitable amount. For example, a composition as described herein may comprise a relatively large amount of one surfactant and a relatively small amount of the other(s), or may comprise relatively equal amounts.

### Co-Surfactants

The compositions described herein typically additionally comprise one or more surfactants as co-surfactants. The one or more co-surfactants may be selected from oleoyl polyoxyl-6-glyceride surfactants (e.g., LABRAFIL® M 1944 CS); linoleoyl macrogolglyceride and linoleoyl polyoxylglyceride surfactants (e.g., LABRAFIL® M 2125 CS); lauroyl macrogolglyceride and lauroyl polyoxylglyceride surfactants (e.g., LABRAFIL® M 2130 CS); propylene glycol monocaprylate Type II surfactants (e.g., CAPMUL® PG-8), glycerol monocaprylate Type I (e.g., CAPMUL® MCM) surfactants, and propylene glycol monolaurate Type II surfactants (e.g., CAPMUL® PG-12). The one or more co-surfactant(s) can be present in a composition as described herein in any suitable amount. For example, a composition as described herein may include one or more of these co-surfactants in a total amount of from about 5% to about 30% w/w, or from about 5% to about 25% w/w, including about 5% to about 20% w/w, including about 5% w/w, about 6% w/w, about 7% w/w, about 8% w/w, about 9% w/w, about 10% w/w, about 11% w/w, about 12% w/w, about 13% w/w, about 14% w/w, about 15% w/w, about 16% w/w, about 17% w/w, about 18% w/w, about 19% w/w, about 20% w/w, and any value therebetween. When combinations of co-surfactants are used, they each may be present in any suitable amount. For example, a composition as described herein may comprise a relatively large amount of one co-surfactant and a relatively small amount of the other(s), or may comprise relatively equal amounts.

In specific embodiments, a composition as described herein comprises a tocopherol polyethylene glycol succinate surfactant (e.g., Vitamin E TPGS). For example, a composition as described herein may include Vitamin E TPGS in an amount of from about 5% w/w to about 55% w/w, including about 6% w/w, or about 14% w/w to about 17% w/w, including about 14% w/w, about 15% w/w, about 16% w/w, or about 17% w/w. In other specific embodiments, a composition as described herein comprises a tocopherol polyethylene glycol succinate surfactant (e.g., Vitamin E TPGS) and a polyoxyethylene castor oil surfactant (e.g., PEG-35 castor oil, such as KOLLIPHOR® ELP), such as comprising Vitamin E TPGS and PEG 35 castor oil (e.g., KOLLIPHOR® ELP), in a total amount of from about 5% w/w to about 55% w/w, such as about 12% w/w or about 18% w/w Vitamin E TPGS, and about 21% w/w or about 30% w/w PEG 35 castor oil (e.g., KOLLIPHOR® ELP).

In accordance with any of these embodiments, the composition may further comprise a co-surfactant, such as an oleoyl polyoxyl-6-glyceride surfactant (e.g., LABRAFIL® M 1944 CS), such as in an amount of from about 5% w/w to about 30% w/w or from about 5% w/w to about 25% w/w. For example, a composition as described herein may comprise an oleoyl polyoxyl-6-glyceride surfactant (e.g., LABRAFIL® M 1944 CS) in an amount of about 12% w/w, about 13% w/w, about 15% w/w, or about 17% w/w. In specific examples, a composition as described herein may include Vitamin E TPGS in an amount of from about 5% w/w to about 55% w/w, including about 6% w/w, and an oleoyl polyoxyl-6-glyceride surfactant (e.g., LABRAFIL® M 1944 CS) as a co-surfactant, in an amount of from about 5% w/w to about 30% w/w or from about 5% w/w to about 25% w/w, including about 17% w/w. Alternatively, a composition as described herein may include Vitamin E TPGS and PEG 35 castor oil (e.g., KOLLIPHOR® ELP) in a total amount of from about 5% w/w to about 55% w/w, such as about 12% w/w Vitamin E TPGS, and about 21% PEG 35 castor oil (e.g., KOLLIPHOR® ELP), and an oleoyl polyoxyl-6-glyceride surfactant (e.g., LABRAFIL® M 1944 CS) as a co-surfactant in an amount of from about 5% w/w to about 30% w/w or from about 5% w/w to about 25% w/w, including about 13% w/w. Alternatively, a composition as described herein may include Vitamin E TPGS and PEG 35 castor oil (e.g., KOLLIPHOR® ELP) in a total amount of from about 5% w/w to about 55% w/w, such as about 18% w/w Vitamin E TPGS, and about 30% PEG 35 castor oil (e.g., KOLLIPHOR® ELP), and an oleoyl polyoxyl-6-glyceride surfactant (e.g., LABRAFIL® M 1944 CS) as a co-surfactant in an amount of from about 5% w/w to about 30% w/w or from about 5% w/w to about 25% w/w, including about 13% w/w. Alternatively, a composition as described herein may include Vitamin E TPGS and PEG 35 castor oil (e.g., KOLLIPHOR® ELP) in a total amount of from about 5% w/w to about 55% w/w, such as about 17% w/w Vitamin E TPGS and about 28% PEG 35 castor oil (e.g., KOLLIPHOR® ELP), and an oleoyl polyoxyl-6-glyceride surfactant (e.g., LABRAFIL® M 1944 CS) as a co-surfactant in an amount of from about 5% w/w to about 30% w/w or from about 5% w/w to about 25% w/w, including about 15% w/w.

Alternatively, the composition may further comprise a co-surfactant such as a glycerol monocaprylocaprate Type I surfactant (e.g., CAPMUL® MCM), such as in an amount of from about 5% w/w to about 30% w/w or from about 5% w/w to about 25% w/w. For example, a composition as described herein may comprise a glycerol monocaprylocaprate Type I surfactant (e.g., CAPMUL® MCM) as a co-surfactant in an amount of about 7% w/w. In specific examples, a composition as described herein may include Vitamin E TPGS in an amount of from about 5% w/w to about 55% w/w, including about 17% w/w, and a glycerol monocaprylocaprate Type I surfactant (e.g., CAPMUL® MCM) as a co-surfactant in an amount of from about 5% w/w to about 30% w/w or from about 5% w/w to about 25% w/w, including about 7% w/w.

Alternatively, the composition may further comprise a co-surfactant such as a propylene glycol monocaprylate Type II surfactant (e.g., CAPMUL® PG-8), such as in an amount of from about 5% w/w to about 30% w/w or from about 5% w/w to about 25% w/w. For example, a composition as described herein may comprise a propylene glycol monocaprylate Type II surfactant (e.g., CAPMUL® PG-8) as a co-surfactant in an amount of about 7% w/w. In specific examples, a composition as described herein may include Vitamin E TPGS in an amount of from about 5% w/w to about 55% w/w, including about 17% w/w, and a propylene glycol monocaprylate Type II surfactant (e.g., CAPMUL® PG-8) as a co-surfactant in an amount of from about 5% w/w to about 30% w/w or from about 5% w/w to about 25% w/w, including about 7% w/w.

Alternatively, the composition may further comprise a co-surfactant such as a propylene glycol monolaurate Type II surfactant (e.g., CAPMUL® PG-12), such as in an amount of from about 5% w/w to about 30% w/w or from about 5% w/w to about 25% w/w. For example, a composition as described herein may comprise a propylene glycol monolaurate Type II surfactant (e.g., CAPMUL® PG-12) as a co-surfactant in an amount of about 6% w/w or about 7% w/w. In specific examples, a composition as described herein may include Vitamin E TPGS in an amount of from about 5% w/w to about 55% w/w, including about 17% w/w, and a propylene glycol monolaurate Type II surfactant (e.g., CAPMUL® PG-12) as a co-surfactant in an amount of from about 5% w/w to about 30% w/w or from about 5% w/w to about 25% w/w, including about 7% w/w. Alternatively, a composition as described herein may include Vitamin E TPGS in an amount of from about 5% w/w to about 55% w/w, including about 15% w/w, and a propylene glycol monolaurate Type II surfactant (e.g., CAPMUL® PG-12) as a co-surfactant in an amount of from about 5% w/w to about 30% w/w or from about 5% w/w to about 25% w/w, including about 7% w/w. In a further alternative, a composition as described herein may include Vitamin E TPGS in an amount of from about 5% w/w to about 55% w/w, including about 14% w/w to about 17% w/w, including about 14% w/w, about 15% w/w, about 16% w/w, or about 17% w/w, and a propylene glycol monolaurate Type II surfactant (e.g., CAPMUL® PG-12) as a co-surfactant in an amount of from about 5% w/w to about 30% w/w or from about 5% w/w to about 25% w/w, including about 6% w/w or about 7% w/w.

### Precipitation Inhibitors

A composition as described herein optionally may comprise a precipitation inhibitor. Non-limiting examples of suitable precipitation inhibitors include one or more of polyvinyl caprolactam-polyvinyl acetate-polyethylene glycol graft copolymer solubilizers, (e.g., SOLUPLUS®), polyoxyethylene polyoxypropylene glycol solubilizers (e.g., KOLLIPHOR® P188 or P407), polyvinylpyrrolidone solubilizers (e.g., KOLLIDON® K-30), vinylpyrrolidone-vinyl acetate copolymer solubilizers (e.g., KOLLIDON® VA-64), polyvinyl alcohol/polyethylene glycol graft copolymer solubilizers (e.g., KOLLICOAT® IR), hydroxypropyl methylcellulose solubilizers (also known as HMPC and hypromellose), and hypromellose acetate succinate solubilizers (e.g., AQOAT®). A precipitation inhibitor may be used in any suitable amount. For example, a composition may comprise one or more precipitation inhibitors in a total amount of from about 0.5% w/w to about 5% w/w or from about 0.5% w/w to about 1% w/w, including about 0.5% w/w, about 0.75% w/w, about 1% w/w, about 1.5% w/w, about 2% w/w, about 2.5% w/w, about 3% w/w, about 3.5% w/w, about 4% w/w, about 4.5% w/w, about 5% w/w, and any value therebetween. For example, a composition as described herein may comprise a polyvinyl caprolactam-polyvinyl acetate-polyethylene glycol graft copolymer solubilizer (e.g., SOLUPLUS®) in an amount from about 0.5% w/w to about 5% w/w, including about 0.5% w/w, or about 0.65% w/w, or about 0.75% w/w.

### Co-Solvent

The compositions described herein optionally may comprise one or more co-solvents. Non-limiting examples of suitable co-solvents include diethylene glycol monoethyl ether (e.g., TRANSCUTOL® HP or TRANSCUTOL® P), tetrahydrofurfuryl alcohol polyethylene glycol ether (e.g., glycofurol), and N-methyl pyrrolidone (e.g., PHARMASOLVE™ V). The co-solvent may be present in any suitable amount. For example, a composition as described herein may include a co-solvent in an amount of from about 10% w/w to about 35% w/w, including about 10% w/w, about 15% w/w, about 19% w/w, about 20% w/w, about 25% w/w, about 30% w/w, about 35% w/w, and any value therebetween. In some embodiments, a composition as described herein comprises diethylene glycol monoethyl ether as a co-solvent (e.g., TRANSCUTOL® HP or TRANSCUTOL® P), such as in an amount of about 20% w/w or 25% w/w of the composition.

### Solubilizers

The compositions described herein may comprise one or more solubilizers. Non-limiting examples of suitable solubilizers include those having an HLB value of from 11 to 15, such as polyethylene glycol caprylic/capric glycerides solubilizers, such as polyethylene glycol caprylocaproyl polyoxylglyceride and caprylocaproyl macrogoglyceride solubilizers (e.g., ACCONON® MC8-2), polyglyceryl oleate solubilizers (e.g., polyglyceryl-10-oleate such as CAPROL® PGE 860), polyoxyethylene sorbitan monooleate solubilizers (e.g., polyoxyethylene (20) sorbitan monooleate or polysorbate 80). In some embodiments, the composition does not include a caprylocaproyl polyoxyl-8 glyceride solubilizer/surfactant (e.g., does not include LABRASOL® and does not include LABRASOL® ALF). Embodiments excluding caprylocaproyl polyoxyl-8 glyceride solubilizers/surfactants still may (optionally) comprise polyethylene glycol caprylocaproyl polyoxylglyceride/caprylocaproyl macrogoglyceride solubilizers (e.g., ACCONON® MC8 2, a polyethylene glycol caprylic/capric glycerides solubilizer).

When present, the solubilizer may be present in any suitable amount. For example, a composition as described herein may comprise a total amount of one or more solubilizers of from about 10% w/w to about 65% w/w, such as about 15% w/w, about 20% w/w, about 25% w/w, about 27% w/w, about 30% w/w, about 35% w/w, about 40% w/w, about 45% w/w, about 50% w/w, about 55% w/w, about 60% w/w, about 65% w/w, or any value therebetween. When combinations of solubilizers are used, they each may be present in any suitable amount.

A composition as described herein may comprise a solubilizer selected from one or more of polyethylene glycol caprylic/capric glycerides solubilizers, such as polyethylene glycol caprylocaproyl polyoxylglyceride and caprylocaproyl macrogoglyceride solubilizers (e.g., ACCONON® MC8-2), polyglyceryl oleate solubilizers (e.g., polyglyceryl-10-oleate such as CAPROL® PGE 860), polyoxyethylene sorbitan monooleate solubilizers (e.g., polyoxyethylene (20) sorbitan monooleate or polysorbate 80). A solubilizer may be used in relatively large amounts, such as from about 10% to about 65% w/w as disclosed above. For example, a composition as described herein may comprise a polyethylene glycol caprylocaproyl polyoxylglyceride/caprylocaproyl macrogoglyceride solubilizer (e.g., ACCONON® MC8-2), such as in an amount from about 10% to about 65% w/w, including about 25% w/w, about 40% w/w, about 55% w/w, or about 60% w/w.

Thus, for example, in some embodiments, a composition as described herein comprises a combination of a solubilizer and a precipitation inhibitor, such as a polyethylene glycol caprylocaproyl polyoxylglyceride/caprylocaproyl macrogoglyceride solubilizer (e.g., ACCONON® MC8-2) and a polyvinyl caprolactam-polyvinyl acetate-polyethylene glycol graft copolymer precipitation inhibitor (e.g., SOLUPLUS®). For example, a composition as described herein may comprise about 25% w/w, about 40% w/w, about 55% w/w, or about 60% w/w, including about 55% w/w to about 60% w/w, polyethylene glycol caprylocaproyl polyoxylglyceride/caprylocaproyl macrogoglyceride solubilizer (e.g., ACCONON® MC8-2) and from about 0.5% w/w to about 0.75% w/w, including about 0.5% w/w, or about 0.65% w/w, or about 0.75% w/w, polyvinyl caprolactam-polyvinyl acetate-polyethylene glycol graft copolymer solubilizer (e.g., SOLUPLUS®).

### Antioxidants

The compositions described herein typically comprise one or more antioxidants. Non-limiting examples of suitable antioxidants include DL-methionine, butylated hydroxy anisole (BHA), butylated hydroxy toluene (BHT), arginine, cysteine, ascorbic palmitate, sodium metabisulfite, sodium thiosulfate, propyl gallate, gamma linoleic acid, ascorbic acid, ethylenediaminetetraacetic acid (EDTA), and alpha tocopherol. Thus, in accordance with any of the foregoing embodiments, a composition as described herein may comprise an antioxidant. For example, a composition as described herein may comprise one or more selected from DL-methionine, BHA, and BHT. In some embodiments, a composition may comprise one or more of DL-methionine, arginine and cysteine. In some embodiments, a composition may comprise DL-methionine. The antioxidant(s) may be present in any suitable amount. For example, a composition as described herein may comprise one or more antioxidants in a total amount of from about 0.01% to about 1.0% w/w, including from about 0.05% to about 1.0% w/w, including any value therebetween.

### Sweetening Agents Flavoring Agents

The compositions described herein optionally may comprise one or more of sweetening agent(s) and/or one or more flavoring agent(s). Examples of suitable sweetening agents include, but are not limited to, sucralose, glycyrrhizic acid and its salts (e.g., ammonium glycyrrhizinate), saccharin, aspartame, acesulfame potassium, cyclamate, erythritol, and neotame. Thus, in accordance with any of the foregoing embodiments, a composition as described herein may comprise a sweetening agent. For example, a composition as described herein may comprise one or more sweetening agent(s) selected from sucralose and ammonium glycyrrhizinate. The sweetening agent(s) may be present in any suitable amount. For example, a composition as described herein may comprise one or more sweetening agents in a total amount of from about 0.015% w/w to about 0.75% w/w or from about 0.015% w/w to about 2% w/w, including any value therebetween.

Examples of suitable flavoring agents include, but are not limited to, vanilla flavor, banana flavor, grapefruit flavor, strawberry flavor, raspberry flavor, bubble gum flavor, and caramel flavor. Thus, in accordance with any of the foregoing embodiments, a composition as described herein may comprise a flavoring agent. For example, a composition as described herein may comprise vanilla flavor and banana flavor. The flavoring agent(s) may be present in any suitable amount. For example, a composition as described herein may comprise one or more flavoring agents in a total amount of from about 0.01% w/w to about 1.0% w/w, including any value therebetween.

### Water

In some embodiments, a composition as described herein comprises water (e.g., purified water). In some embodiments the water is present in an amount of from about 1% to about 5% w/w, including about 1% w/w, about 1.5% w/w, about 2% w/w, about 2.5% w/w, about 3% w/w, about 3.5% w/w, about 4% w/w, about 4.5% w/w, and 5% w/w.

### Additional Components

A composition as described herein optionally may further comprise one or more additional pharmaceutically acceptable components suitable for use in an oral liquid pharmaceutical composition. For example, a composition as described herein optionally may further comprise one or more pharmaceutically acceptable excipients such as viscosity adjusting agents (e.g., thickeners), diluents, pH adjusting agents, colorants, other flavoring agents, other taste-masking agents, other preservatives, etc.

### Excluded Components

As mentioned above, WO 2018/037310 discloses enzalutamide compositions that include large amounts of LABRASOL® and propylene glycol. In particular, WO 2018/037310 discloses enzalutamide compositions that include one or more of LABRASOL®, LABRASOL® ALF, polyethylene glycol 300 (PEG 300) and polyethylene glycol 400 (PEG 400) as “surfactants”, and also discloses enzalutamide compositions that include propylene glycol and/or ethanol as a solvent. The compositions of the present disclosure do not require such components. Thus, in specific embodiments of any of the embodiments disclosed herein, the composition does not include caprylocaproyl polyoxyl-8 glyceride surfactants/solubilizers as disclosed in WO 2018/037310 (e.g., LABRASOL®, LABRASOL® ALF, which WO 2018/037310 refers to as surfactants but also may be considered to be solubilizers) and does not include polyethylene glycol (e.g., PEG 300, PEG 400). In further specific embodiments of any of the embodiments disclosed herein, the composition additionally or alternatively does not include propylene glycol and does not include ethanol. That is, in specific embodiments of any of the embodiments disclosed herein, the composition does not include any of caprylocaproyl polyoxyl-8 glyceride surfactants/solubilizers (e.g., LABRASOL®, LABRASOL® ALF), and does not include polyethylene glycol (e.g., PEG 300, PEG 400). In further specific embodiments of any of the embodiments disclosed herein, the composition does not include propylene glycol and does not include ethanol. In further specific embodiments of any of the embodiments disclosed herein, the composition does not include any of caprylocaproyl polyoxyl-8 glyceride surfactants/solubilizers (e.g., LABRASOL®, LABRASOL® ALF), propylene glycol, and ethanol. In other specific embodiments of any of the embodiments disclosed herein, the composition does not include amounts of caprylocaproyl polyoxyl-8 glyceride surfactants/solubilizers (e.g., LABRASOL®, LABRASOL® ALF), polyethylene glycol (e.g., PEG 300, PEG 400), propylene glycol, or ethanol used in the compositions of WO 2018/037310. For example, in specific embodiments of any of the embodiments disclosed herein, the composition does not include from about 30% to about 90% w/w of one or more of caprylocaproyl polyoxyl-8 glyceride surfactants/solubilizers and polyethylene glycol. In further specific embodiments of any of the embodiments disclosed herein, the composition additionally or alternatively does not include propylene glycol and does not include ethanol. As noted above, embodiments excluding caprylocaproyl polyoxyl-8 glyceride solubilizers/surfactants (e.g., LABRASOL®, LABRASOL® ALF), still may (optionally) comprise polyethylene glycol caprylic/capric glyceride solubilizers (e.g., ACCONON® MC8-2, a polyethylene glycol caprylic/capric glyceride solubilizer).

### Compositions

The following are disclosed as specific illustrative embodiments of compositions as described herein.

Provided in one aspect are oral liquid pharmaceutical compositions of enzalutamide, comprising:
(i) about 2.5% to about 6% w/w of enzalutamide;
(ii) optionally, about 0.5% to about 25% w/w of an oil selected from one or more of a vegetable oil, a flavored oil, and an essential oil;
(iii) about 5% to about 85% w/w of one or both of a surfactant and a solubilizer, wherein the surfactant comprises one or more selected from tocopherol polyethylene glycol succinate surfactants, polyoxyethylene castor oil surfactants, polyoxyl hydrogenated castor oil surfactants, polyoxyl hydroxy stearate surfactants, lauroyl polyoxylglyceride and lauroyl macrogoglyceride surfactants, stearoyl polyoxylglyceride and stearoyl macrogoglyceride surfactants, and polyoxyl stearate surfactants, and the solubilizer comprises one or more selected from polyethylene glycol caprylic/capric glyceride solubilizers, polyglyceryl oleate solubilizers, and polyoxyethylene sorbitan monooleate solubilizers;
(iv) about 5% to about 25% w/w of a co-surfactant selected from one or more of propylene glycol monolaurate Type II surfactants, oleoyl polyoxyl-6-glyceride surfactants, linoleoyl macrogolglyceride and linoleoyl polyoxylglyceride surfactants, lauroyl macrogolglyceride and lauroyl polyoxylglyceride surfactants, glycerol monocaprylocaprate Type I surfactants, and propylene glycol monocaprylate Type II surfactants;
(v) about 0.01% to about 1% w/w of an antioxidant comprising one or more selected from DL-methionine, butylated hydroxy anisole (BHA), butylated hydroxy toluene (BHT), arginine, cysteine, ascorbic palmitate, sodium metabisulfite, sodium thiosulfate, propyl gallate, gamma linolenic acid, ethylenediaminetetraacetic acid (EDTA), ascorbic acid, and alpha-tocopherol;
(vi) optionally, about 0.5% to about 5% w/w of precipitation inhibitor selected one or more of polyvinyl caprolactam-polyvinyl acetate-polyethylene glycol graft copolymer solubilizers, polyoxyethylene polyoxypropylene glycol solubilizers, polyvinylpyrrolidone solubilizers, vinylpyrrolidone-vinyl acetate copolymer solubilizers, polyvinyl alcohol/polyethylene glycol graft copolymer solubilizers, hydroxypropyl methylcellulose solubilizers, and hypromellose acetate succinate solubilizers; and
(vii) optionally, water (such as about 1% to about 5% w/w water).

Provided in another aspect are oral liquid pharmaceutical compositions of enzalutamide, comprising:
(i) about 2.5% to about 6% w/w of enzalutamide;
(ii) optionally, about 0.5% to about 25% w/w of an oil selected from one or more of a vegetable oil, a flavored oil, and an essential oil;
(iii) about 5% to about 65% w/w of a surfactant, wherein the surfactant comprises one or more selected from tocopherol polyethylene glycol succinate surfactants (e.g., D-α-tocopherol polyethylene glycol 1000 succinate, also known as Vitamin E TPGS and tocophersolan); polyoxyethylene castor oil surfactants (e.g., polyoxyethylene 35 castor oil, also known as PEG-35 castor oil and macrogolglycerol ricinoleate, such as KOLLIPHOR® EL and KOLLIPHOR® ELP); polyoxyl hydrogenated castor oil surfactants (e.g., polyoxyl 40 hydrogenated castor oil, also known as macrogolglycerol hydroxystearate, such as KOLLIPHOR® RH40); polyoxyl hydroxy stearate surfactants (e.g., polyoxyl 15 hydroxy stearate, also known as macrogol (15)-hydroxystearate, polyethylene glycol (15)-hydroxystearate, and polyoxyethylated 12-hydroxystearic acid, such as KOLLIPHOR® HS 15); lauroyl polyoxylglyceride and lauroyl macrogoglyceride surfactants (e.g., GELUCIRER 44/14 and ACCONON® C-44); stearoyl polyoxylglyceride and stearoyl macrogoglyceride surfactants (e.g., GELUCIRE® 50/13 and ACCONON® C-50); and polyoxyl stearate surfactants (e.g., polyoxyl stearate Type I such as GELUCIRE® 48/16);
(iv) about 5% to about 30% w/w of a co-surfactant selected from one or more of propylene glycol monolaurate Type II surfactants (e.g., CAPMUL® PG-12), oleoyl polyoxyl-6-glyceride surfactants (e.g., LABRAFIL® M 1944 CS), linoleoyl macrogolglyceride and linoleoyl polyoxylglyceride surfactants (e.g., LABRAFIL® M 2125 CS), lauroyl macrogolglyceride and lauroyl polyoxylglyceride surfactants (e.g., LABRAFIL® M 2130 CS), propylene glycol monocaprylate Type II (e.g., CAPMUL® PG-8) surfactants, and glycerol monocaprylate Type I surfactants (e.g., CAPMUI® MCM);
(v) about 0.01% to about 1% w/w of an antioxidant comprising one or more selected from DL-methionine, butylated hydroxy anisole (BHA), butylated hydroxy toluene (BHT), arginine, cysteine, ascorbic palmitate, sodium metabisulfite, sodium thiosulfate, propyl gallate, gamma linolenic acid, ethylenediaminetetraacetic acid (EDTA), ascorbic acid, and alpha-tocopherol;
(vi) optionally, about 0.5% to about 5% w/w of precipitation inhibitor selected one or more of polyvinyl caprolactam-polyvinyl acetate-polyethylene glycol graft copolymer solubilizers (e.g., SOLUPLUS®), polyoxyethylene polyoxypropylene glycol solubilizers (e.g., KOLLIPHOR® P 188 or P407), polyvinylpyrrolidone solubilizers (e.g., KOLLIDON® K-30), vinylpyrrolidone-vinyl acetate copolymer solubilizers (e.g., KOLLIDON® VA-64), polyvinyl alcohol/polyethylene glycol graft copolymer solubilizers (e.g., KOLLICOAT® IR), HMPC solubilizers and hypromellose acetate succinate solubilizers (e.g., AQOAT®), and
(vii) optionally, about 1% to about 5% w/w water.

When present, the oil may comprise about 0.5% to about 20% w/w of the composition, optionally wherein the composition comprises about 0.5% to about 5% w/w of the flavored oil or essential oil and about 3% to about 15% w/w of the vegetable oil. Additionally or alternatively, the composition may comprise about 5% to about 50% w/w of the surfactant. Additionally or alternatively, the composition may comprise about 5% to about 20% w/w of the co-surfactant. Additionally or alternatively, the composition optionally may comprise about 0.5% to about 1% w/w of the precipitation inhibitor.

As noted above, a composition as described herein may comprise a combination of surfactants, such as a combination of any two or more of the surfactants listed in (iii) above. In some embodiments comprising a combination of surfactants, one of the surfactants is a polyoxyethylene castor oil surfactant (e.g., PEG-35 castor oil such as KOLLIPHOR® ELP). For example, a composition as described herein may comprise a tocopherol polyethylene glycol succinate surfactant (e.g., Vitamin E TPGS) and a polyoxyethylene castor oil surfactant (e.g., PEG-35 castor oil, such as KOLLIPHOR® ELP). Alternatively, a composition as described herein may comprise a single type of surfactant, such as a tocopherol polyethylene glycol succinate surfactant (e.g., Vitamin E TPGS).

In addition to one or more surfactants, a composition described herein further comprises a co-surfactant. For example, a composition may comprise a tocopherol polyethylene glycol succinate surfactant (e.g., Vitamin E TPGS), a polyoxyethylene castor oil surfactant (e.g., PEG-35 castor oil, such as KOLLIPHOR® ELP), and an oleoyl polyoxyl-6-glyceride co-surfactant (e.g., LABRAFIL® M 1944 CS). In another example, a composition may comprise a tocopherol polyethylene glycol succinate surfactant (e.g., Vitamin E TPGS) and an oleoyl polyoxyl-6-glyceride co-surfactant (e.g., LABRAFIL® M 1944 CS). In another example, a composition may comprise a tocopherol polyethylene glycol succinate surfactant (e.g., Vitamin E TPGS) and a glycerol monocaprylocaprate Type I co-surfactant (e.g., CAPMUL® MCM). In another example, a composition may comprise a tocopherol polyethylene glycol succinate surfactant (e.g., Vitamin E TPGS) and a propylene glycol monocaprylate Type I co-surfactant (e.g., CAPMUL® PG-8). In another example, a composition may comprise a tocopherol polyethylene glycol succinate surfactant (e.g., Vitamin E TPGS) and a propylene glycol monolaurate Type II co-surfactant (e.g., CAPMUL® PG-12).

In some embodiments, the composition further comprises about 10% to about 35% w/w of a co-solvent selected from one or more of diethylene glycol monoethyl ether (e.g., TRANSCUTOL® HP or TRANSCUTOL® P), tetrahydrofurfuryl alcohol polyethylene glycol ether (e.g., glycofurol), and N-methyl pyrrolidone (e.g., PHARMASOLVE™ V).

In some embodiments, the composition comprises or further comprises about 10% to about 65% w/w of a solubilizer comprising one or more selected from polyethylene glycol caprylic/capric glyceride solubilizers, such as polyethylene glycol caprylocaproyl polyoxylglyceride and caprylocaproyl macrogoglyceride solubilizers (e.g., ACCONON® MC8-2), polyglyceryl oleate solubilizers (e.g., polyglyceryl-10-oleate such as CAPROL® PGE 860), and polyoxyethylene sorbitan monooleate solubilizers (e.g., polyoxyethylene (20) sorbitan monooleate or polysorbate 80).

In any embodiments, the oil may comprise peppermint oil as a flavored oil and corn oil as a vegetable oil; the surfactant may be a tocopherol polyethylene glycol succinate surfactant (such as vitamin E TPGS); the co-surfactant may be an oleoyl polyoxyl-6-glyceride surfactant (e.g., LABRAFIL® M 1944 CS), and the precipitation inhibitor may be a polyvinyl caprolactam-polyvinyl acetate-polyethylene glycol graft copolymer solubilizer (e.g., SOLUPLUS®).

Alternatively, the oil may comprise peppermint oil as a flavored oil and corn oil as a vegetable oil; the surfactant may be a tocopherol polyethylene glycol succinate surfactant (such as vitamin E TPGS) and a polyoxyethylene castor oil surfactant (e.g., PEG-35 castor oil such as KOLLIPHOR® ELP); the co-surfactant may be an oleoyl polyoxyl-6-glyceride surfactant (e.g., LABRAFIL® M 1944 CS), and the precipitation inhibitor may be a polyvinyl caprolactam-polyvinyl acetate-polyethylene glycol graft copolymer solubilizer (e.g., SOLUPLUS®).

Alternatively, the oil may comprise peppermint oil as a flavored oil and corn oil as a vegetable oil; the surfactant may be a tocopherol polyethylene glycol succinate surfactant (such as vitamin E TPGS); the co-surfactant may be a propylene glycol monocaprylate Type II surfactant (e.g., CAPMUL® PG-8), and the precipitation inhibitor may be a polyvinyl caprolactam-polyvinyl acetate-polyethylene glycol graft copolymer solubilizer (e.g., SOLUPLUS®).

Alternatively, the oil may comprise peppermint oil as a flavored oil and corn oil as a vegetable oil; the surfactant may be a tocopherol polyethylene glycol succinate surfactant (such as vitamin E TPGS); the co-surfactant may be a propylene glycol monolaurate Type II surfactant (e.g., CAPMUL® PG-8), and the precipitation inhibitor may be a polyvinyl caprolactam-polyvinyl acetate-polyethylene glycol graft copolymer solubilizer (e.g., SOLUPLUS®).

Alternatively, the oil may comprise peppermint oil as a flavored oil and corn oil as a vegetable oil; the surfactant may be a tocopherol polyethylene glycol succinate surfactant (such as vitamin E TPGS); the co-surfactant may be a propylene glycol monolaurate Type II surfactant (such as CAPMUL® PG-12), and the precipitation inhibitor may be a polyvinyl caprolactam-polyvinyl acetate-polyethylene glycol graft copolymer solubilizer (e.g., SOLUPLUS®).

In some embodiments, the composition comprises:
(i) about 2.5% w/w of enzalutamide;
(ii) about 5% w/w of a flavored oil;
(iii) about 10% w/w of a vegetable oil;
(iv) about 7% w/w of the propylene glycol monolaurate Type II co-surfactant;
(v) about 15% w/w of the tocopherol polyethylene glycol succinate surfactant;
(vi) about 60% w/w of the polyethylene glycol caprylic/capric glyceride solubilizer;
(vii) about 0.5% w/w of the polyvinyl caprolactam-polyvinyl acetate-polyethylene glycol graft copolymer precipitation inhibitor;
(viii) an antioxidant;
(ix) optionally, a sweetening agent;
(x) optionally, a flavoring agent; and
(xi) water in an amount of about 1.5% w/w.

In specific embodiments of the foregoing, the flavored oil is peppermint oil; the vegetable oil is corn oil; the propylene glycol monolaurate Type II co-surfactant is CAPMUL® PG-12; the tocopherol polyethylene glycol succinate surfactant is D-α-tocopherol polyethylene glycol 1000 succinate surfactant; the polyethylene glycol caprylic/capric glycerides solubilizer is ACCONON® MC8-2; the polyvinyl caprolactam-polyvinyl acetate-polyethylene glycol graft copolymer precipitation inhibitor is SOLUPLUS®; and the antioxidant comprises one or more selected from DL methionine, BHA, and BHT. In further specific embodiments, the sweetening agent is present and comprises one or more selected from sucralose and ammonium glycyrrhizinate; and the flavoring agent is present and comprises vanilla and banana.

In other specific embodiments, the composition comprises:
(i) about 2.5% w/w of enzalutamide;
(ii) about 5% w/w of a flavored oil;
(iii) about 10% w/w of a vegetable oil;
(iv) about 7% w/w of the propylene glycol monolaurate Type II co-surfactant;
(v) about 15% w/w of the tocopherol polyethylene glycol succinate surfactant;
(vi) about 60% w/w of the polyethylene glycol caprylic/capric glyceride solubilizer;
(vii) about 0.5% w/w of the polyvinyl caprolactam-polyvinyl acetate-polyethylene glycol graft copolymer precipitation inhibitor;
(viii) an antioxidant;
(ix) optionally, a sweetening agent;
(x) optionally, a flavoring agent; and
(xi) water in an amount of about 3% w/w.

In specific embodiments of the foregoing, the flavored oil is peppermint oil; the vegetable oil is corn oil; the propylene glycol monolaurate Type II co-surfactant is CAPMUL® PG-12; the tocopherol polyethylene glycol succinate surfactant is D-α-tocopherol polyethylene glycol 1000 succinate surfactant; the polyethylene glycol caprylic/capric glycerides solubilizer is ACCONON® MC8-2; the polyvinyl caprolactam-polyvinyl acetate-polyethylene glycol graft copolymer precipitation inhibitor is SOLUPLUS®; and the antioxidant comprises DL methionine. In further specific embodiments, the sweetening agent is present and comprises one or more selected from sucralose and ammonium glycyrrhizinate; and the flavoring agent is present and comprises vanilla and banana.

In other specific embodiments, the composition comprises:
(i) about 2.5% w/w of enzalutamide;
(ii) about 5% w/w of a flavored oil;
(iii) about 10% w/w of a vegetable oil;
(iv) about 7% w/w of the propylene glycol monolaurate Type II co-surfactant;
(v) about 15% w/w of the tocopherol polyethylene glycol succinate surfactant;
(vi) about 60% w/w of the polyethylene glycol caprylic/capric glyceride solubilizer;
(vii) about 0.5% w/w of the polyvinyl caprolactam-polyvinyl acetate-polyethylene glycol graft copolymer precipitation inhibitor;
(viii) an antioxidant;
(ix) optionally, a sweetening agent;
(x) optionally, a flavoring agent; and
(xi) water in an amount of about 1.5% w/w.

In specific embodiments of the foregoing, the flavored oil is peppermint oil; the vegetable oil is corn oil; the propylene glycol monolaurate Type II co-surfactant is CAPMUL® PG-12; the tocopherol polyethylene glycol succinate surfactant is D-α-tocopherol polyethylene glycol 1000 succinate surfactant; the polyethylene glycol caprylic/capric glycerides solubilizer is ACCONON® MC8-2; the polyvinyl caprolactam-polyvinyl acetate-polyethylene glycol graft copolymer precipitation inhibitor is SOLUPLUS®; and the antioxidant comprises DL methionine. In further specific embodiments, the sweetening agent is present and comprises one or more selected from sucralose and ammonium glycyrrhizinate; and the flavoring agent is present and comprises vanilla and banana.

In other specific embodiments, the composition comprises:
(i) about 2.5% w/w of enzalutamide;
(ii) about 5% w/w of a flavored oil;
(iii) about 10% w/w of a vegetable oil;
(iv) about 7% w/w of the propylene glycol monolaurate Type II co-surfactant;
(v) about 15% w/w of the tocopherol polyethylene glycol succinate surfactant;
(vi) about 60% w/w of the polyethylene glycol caprylic/capric glyceride solubilizer;
(vii) an antioxidant;
(viii) optionally, a sweetening agent;
(ix) optionally, a flavoring agent; and
(x) water in an amount of about 3% w/w.

In specific embodiments of the foregoing, the flavored oil is peppermint oil; the vegetable oil is corn oil; the propylene glycol monolaurate Type II co-surfactant is CAPMUL® PG-12; the tocopherol polyethylene glycol succinate surfactant is D-α-tocopherol polyethylene glycol 1000 succinate surfactant; the polyethylene glycol caprylic/capric glycerides solubilizer is ACCONON® MC8-2; and the antioxidant comprises DL methionine. In further specific embodiments, the sweetening agent is present and comprises one or more selected from sucralose and ammonium glycyrrhizinate; and the flavoring agent is present and comprises vanilla and banana.

In other specific embodiments, the composition comprises:
(i) about 2.5% w/w of enzalutamide;
(ii) about 7% w/w of the propylene glycol monolaurate Type II co-surfactant;
(iii) about 15% w/w of the tocopherol polyethylene glycol succinate surfactant;
(iv) about 60% w/w of the polyethylene glycol caprylic/capric glyceride solubilizer;
(v) about 0.5% w/w of the polyvinyl caprolactam-polyvinyl acetate-polyethylene glycol graft copolymer precipitation inhibitor;
(vi) an antioxidant;
(vii) optionally, a sweetening agent;
(viii) optionally, a flavoring agent; and
(ix) water in an amount of about 3% w/w.

In specific embodiments of the foregoing, the propylene glycol monolaurate Type II co-surfactant is CAPMUL® PG-12; the tocopherol polyethylene glycol succinate surfactant is D-α-tocopherol polyethylene glycol 1000 succinate surfactant; the polyethylene glycol caprylic/capric glycerides solubilizer is ACCONON® MC8-2; the polyvinyl caprolactam-polyvinyl acetate-polyethylene glycol graft copolymer precipitation inhibitor is SOLUPLUS®; and the antioxidant comprises DL methionine. In further specific embodiments, the sweetening agent is present and comprises one or more selected from sucralose and ammonium glycyrrhizinate; and the flavoring agent is present and comprises vanilla and banana.

In other specific embodiments, the composition comprises:
(i) about 2.5% w/w of enzalutamide;
(ii) about 5% w/w of a flavored oil;
(iii) about 10% w/w of a vegetable oil;
(iv) about 7% w/w of the propylene glycol monolaurate Type II co-surfactant;
(v) about 15% w/w of the tocopherol polyethylene glycol succinate surfactant;
(vi) about 60% w/w of the polyethylene glycol caprylic/capric glyceride solubilizer;
(vii) about 0.5% w/w of the polyvinyl caprolactam-polyvinyl acetate-polyethylene glycol graft copolymer precipitation inhibitor;
(viii) an antioxidant;
(ix) optionally, a sweetening agent;
(x) optionally, a flavoring agent; and
(xi) water in an amount of about 3% w/w.

In specific embodiments of the foregoing, the flavored oil is peppermint oil; the vegetable oil is corn oil; the propylene glycol monolaurate Type II co-surfactant is CAPMUL® PG-12; the tocopherol polyethylene glycol succinate surfactant is D-α-tocopherol polyethylene glycol 1000 succinate surfactant; the polyethylene glycol caprylic/capric glycerides solubilizer is ACCONON® MC8-2; the polyvinyl caprolactam-polyvinyl acetate-polyethylene glycol graft copolymer precipitation inhibitor is SOLUPLUS®; and the antioxidant comprises arginine or cysteine. In further specific embodiments, the sweetening agent is present and comprises one or more selected from sucralose and ammonium glycyrrhizinate; and the flavoring agent is present and comprises vanilla and banana.

In other specific embodiments, the composition comprises:
(i) about 2.5% w/w of enzalutamide;
(ii) about 5% w/w of a flavored oil;
(iii) about 10% w/w of a vegetable oil;
(iv) about 7% w/w of the propylene glycol monolaurate Type II co-surfactant;
(v) about 15% w/w of the tocopherol polyethylene glycol succinate surfactant;
(vi) about 60% w/w of the polyethylene glycol caprylic/capric glyceride solubilizer;
(vii) about 0.5% w/w of the polyvinyl caprolactam-polyvinyl acetate-polyethylene glycol graft copolymer precipitation inhibitor;
(viii) an antioxidant;
(ix) optionally, a sweetening agent;
(x) optionally, a flavoring agent; and
(xi) water in an amount of about 1.5% w/w.

In specific embodiments of the foregoing, the flavored oil is peppermint oil; the vegetable oil is corn oil; the propylene glycol monolaurate Type II co-surfactant is CAPMUL® PG-12; the tocopherol polyethylene glycol succinate surfactant is D-α-tocopherol polyethylene glycol 1000 succinate surfactant; the polyethylene glycol caprylic/capric glycerides solubilizer is ACCONON® MC8-2; the polyvinyl caprolactam-polyvinyl acetate-polyethylene glycol graft copolymer precipitation inhibitor is SOLUPLUS®; and the antioxidant comprises DL methionine. In further specific embodiments, the sweetening agent is present and comprises one or more selected from sucralose and ammonium glycyrrhizinate; and the flavoring agent is present and comprises vanilla and banana.

In other specific embodiments, the oral liquid pharmaceutical composition comprises:
(i) about 3% w/w of enzalutamide;
(ii) about 1.0% w/w of a flavored oil;
(iii) about 3.5% w/w of a vegetable oil;
(iv) about 13% w/w of a oleoyl polyoxyl-6-glyceride co-surfactant, such as LABRAFIL® M 1944 CS;
(v) about 12% w/w of a tocopherol polyethylene glycol succinate surfactant, such as Vitamin E TPGS;
(vi) about 20% w/w of a diethylene glycol monoethyl ether co-solvent, such as TRANSCUTOL® HP or TRANSCUTOL® P;
(vii) about 26% w/w of a polyethylene glycol caprylic/capric glyceride solubilizer, such as caprylocaproyl polyoxylglyceride and caprylocaproyl macrogoglyceride solubilizers, such as ACCONON® MC8-2;
(viii) about 0.75% w/w of a polyvinyl caprolactam-polyvinyl acetate-polyethylene glycol graft copolymer precipitation inhibitor, such as SOLUPLUS®;
(ix) about 21% w/w of a polyoxyethylene castor oil surfactant, such as PEG-35 castor oil, such as KOLLIPHOR® ELP;
(x) an antioxidant;
(xi) optionally, a sweetening agent; and
(xii) optionally, a flavouring agent.

In further specific embodiments of the foregoing, the flavored oil is peppermint oil; the vegetable oil is corn oil; the oleoyl polyoxyl-6-glyceride co-surfactant is LABRAFIL® M 1944 CS; the tocopherol polyethylene glycol succinate surfactant is Vitamin E TPGS; the diethylene glycol monoethyl ether co-solvent is TRANSCUTOL® HP; the polyethylene glycol caprylic/capric glycerides solubilizer is ACCONON® MC8-2; the polyvinyl caprolactam-polyvinyl acetate-polyethylene glycol graft copolymer precipitation inhibitor is SOLUPLUS®; the polyoxyethylene castor oil surfactant is KOLLIPHOR® ELP; the antioxidant and comprises one or more selected from butylated hydroxy anisole (BHA) and butylated hydroxy toluene (BHT); the sweetening agent is present and comprises one or more selected from sucralose and ammonium glycyrrhizinate; and the flavoring agent is present and comprises vanilla.

In some embodiments, an oral liquid pharmaceutical composition of enzalutamide as described herein comprises:
(i) about 4% w/w of enzalutamide;
(ii) about 1.5% w/w of a flavored oil;
(iii) about 5% w/w of a vegetable oil;
(iv) about 17% w/w of a oleoyl polyoxyl-6-glyceride surfactant, such as LABRAFIL® M 1944 CS;
(v) about 6% w/w of a tocopherol polyethylene glycol succinate surfactant, such as D-α-tocopherol polyethylene glycol 1000 succinate surfactant (Vitamin E TPGS);
(vi) about 25% w/w of a diethylene glycol monoethyl ether co-solvent, such as TRANSCUTOL® HP or TRANSCUTOL® P;
(vii) about 40% w/w of a polyethylene glycol caprylic/capric glyceride solubilizer, such as a caprylocaproyl polyoxylglyceride and caprylocaproyl macrogoglyceride solubilizer or ACCONON® MC8-2;
(viii) about 0.5% w/w of a polyvinyl caprolactam-polyvinyl acetate-polyethylene glycol graft copolymer precipitation inhibitor, such as SOLUPLUS®;
(ix) an antioxidant;
(x) optionally, a sweetening agent; and
(xi) optionally, a flavoring agent.

In further specific embodiments of the foregoing, the flavored oil is peppermint oil; the vegetable oil is corn oil; the oleoyl polyoxyl-6-glyceride surfactant is LABRAFIL® M 1944 CS; the tocopherol polyethylene glycol succinate surfactant is Vitamin E TPGS; the diethylene glycol monoethyl ether co-solvent is TRANSCUTOL® HP; the polyethylene glycol caprylic/capric glycerides solubilizer is ACCONON® MC8-2; the polyvinyl caprolactam-polyvinyl acetate-polyethylene glycol graft copolymer precipitation inhibitor is SOLUPLUS®; the antioxidant comprises one or more selected from butylated hydroxy anisole (BHA) and butylated hydroxy toluene (BHT); the sweetening agent is present and comprises one or more selected from sucralose and ammonium glycyrrhizinate; and the flavoring agent is present and comprises vanilla.

In specific embodiments, the oral liquid pharmaceutical composition comprises:
(i) about 3% w/w of enzalutamide;
(ii) about 5% w/w of a flavored oil;
(iii) about 12% w/w of a vegetable oil;
(iv) about 13% w/w of a oleoyl polyoxyl-6-glyceride co-surfactant, such as LABRAFIL® M 1944 CS;
(v) about 18% w/w of a tocopherol polyethylene glycol succinate surfactant, such as Vitamin E TPGS;
(vi) about 20% w/w of a diethylene glycol monoethyl ether co-solvent, such as TRANSCUTOL® HP or TRANSCUTOL® P;
(vii) about 0.75% w/w of a polyvinyl caprolactam-polyvinyl acetate-polyethylene glycol graft copolymer precipitation inhibitor, such as SOLUPLUS®;
(viii) about 30% w/w of a polyoxyethylene castor oil surfactant, such as PEG-35 castor oil, such as KOLLIPHOR® ELP;
(x) an antioxidant;
(xi) optionally, a sweetening agent; and
(xii) optionally, a flavouring agent.

In specific embodiments, the flavored oil is peppermint oil; the vegetable oil is corn oil; the oleoyl polyoxyl-6-glyceride co-surfactant is LABRAFIL® M 1944 CS; the tocopherol polyethylene glycol succinate surfactant is Vitamin E TPGS; the diethylene glycol monoethyl ether co-solvent is TRANSCUTOL® HP; the polyvinyl caprolactam-polyvinyl acetate-polyethylene glycol graft copolymer precipitation inhibitor is SOLUPLUS®; the polyoxyethylene castor oil surfactant is KOLLIPHOR® ELP; the antioxidant comprises one or more selected from butylated hydroxy anisole (BHA) and butylated hydroxy toluene (BHT); the sweetening agent is present and comprises one or more selected from sucralose and ammonium glycyrrhizinate; and the flavoring agent is present and comprises vanilla.

In some embodiments, an oral liquid pharmaceutical composition of enzalutamide as described herein comprises:
(i) about 3% w/w of enzalutamide;
(ii) about 5% w/w of a flavored oil;
(iii) about 11% w/w of a vegetable oil;
(iv) about 7% w/w of a glycerol monocaprylocaprate Type I co-surfactant, such as CAPMUL® MCM;
(v) about 17% w/w of a tocopherol polyethylene glycol succinate surfactant, such as D-α-tocopherol polyethylene glycol 1000 succinate surfactant (Vitamin E TPGS);
(vi) about 55% w/w of a polyethylene glycol caprylic/capric glyceride solubilizer, such as a caprylocaproyl polyoxylglyceride and caprylocaproyl macrogoglyceride solubilizer or ACCONON® MC8-2;
(vii) about 0.5% w/w of a polyvinyl caprolactam-polyvinyl acetate-polyethylene glycol graft copolymer precipitation inhibitor, such as SOLUPLUS®;
(viii) an antioxidant;
(ix) optionally, a sweetening agent; and
(x) optionally, a flavoring agent.

In further specific embodiments of the foregoing, the flavored oil is peppermint oil; the vegetable oil is corn oil; the glycerol monocaprylocaprate Type I co-surfactant is CAPMUL® MCM; the tocopherol polyethylene glycol succinate surfactant is Vitamin E TPGS; the polyethylene glycol caprylic/capric glycerides solubilizer is ACCONON® MC8-2; the polyvinyl caprolactam-polyvinyl acetate-polyethylene glycol graft copolymer precipitation inhibitor is SOLUPLUS®; the antioxidant comprises one or more selected from butylated hydroxy anisole (BHA) and butylated hydroxy toluene (BHT); the sweetening agent is present and comprises one or more selected from sucralose and ammonium glycyrrhizinate; and the flavoring agent is present and comprises vanilla.

In some embodiments, an oral liquid pharmaceutical composition of enzalutamide as described herein comprises:
(i) about 3% w/w of enzalutamide;
(ii) about 5% w/w of a flavored oil;
(iii) about 11% w/w of a vegetable oil;
(iv) about 7% w/w of a propylene glycol monocaprylate Type II co-surfactant, such as CAPMUL® PG-8;
(v) about 17% w/w of a tocopherol polyethylene glycol succinate surfactant, such as D-α-tocopherol polyethylene glycol 1000 succinate surfactant (Vitamin E TPGS);
(vi) about 55% w/w of a polyethylene glycol caprylic/capric glyceride solubilizer, such as a caprylocaproyl polyoxylglyceride and caprylocaproyl macrogoglyceride solubilizer or ACCONON® MC8-2;
(vii) about 0.5% w/w of a polyvinyl caprolactam-polyvinyl acetate-polyethylene glycol graft copolymer precipitation inhibitor, such as SOLUPLUS®;
(viii) an antioxidant;
(ix) optionally, a sweetening agent; and
(x) optionally, a flavoring agent.

In further specific embodiments of the foregoing, the flavored oil is peppermint oil; the vegetable oil is corn oil; the propylene glycol monocaprylate Type II co-surfactant is CAPMUL® PG-8; the tocopherol polyethylene glycol succinate surfactant is Vitamin E TPGS; the polyethylene glycol caprylic/capric glycerides solubilizer is ACCONON® MC8-2; the polyvinyl caprolactam-polyvinyl acetate-polyethylene glycol graft copolymer precipitation inhibitor is SOLUPLUS®; the antioxidant comprises one or more selected from butylated hydroxy anisole (BHA) and butylated hydroxy toluene (BHT); the sweetening agent is present and comprises one or more selected from sucralose and ammonium glycyrrhizinate; and the flavoring agent is present and comprises vanilla.

In some embodiments, an oral liquid pharmaceutical composition of enzalutamide as described herein comprises:
(i) about 3% w/w of enzalutamide;
(ii) about 5% w/w of a flavored oil;
(iii) about 11% w/w of a vegetable oil;
(iv) about 7% w/w of a propylene glycol monolaurate Type II co-surfactant, such as CAPMUL® PG-12;
(v) about 17% w/w of a tocopherol polyethylene glycol succinate surfactant, such as D-α-tocopherol polyethylene glycol 1000 succinate surfactant (Vitamin E TPGS);
(vi) about 55% w/w of a polyethylene glycol caprylic/capric glyceride solubilizer, such as a caprylocaproyl polyoxylglyceride and caprylocaproyl macrogoglyceride solubilizer or ACCONON® MC8-2;
(vii) about 0.5% w/w of a polyvinyl caprolactam-polyvinyl acetate-polyethylene glycol graft copolymer precipitation inhibitor, such as SOLUPLUS®;
(viii) an antioxidant;
(ix) optionally, a sweetening agent; and
(x) optionally, a flavoring agent.

In further specific embodiments of the foregoing, the flavored oil is peppermint oil; the vegetable oil is corn oil; the propylene glycol monolaurate Type II co-surfactant is CAPMUL® PG-12; the tocopherol polyethylene glycol succinate surfactant is Vitamin E TPGS; the polyethylene glycol caprylic/capric glycerides solubilizer is ACCONON® MC8-2; the polyvinyl caprolactam-polyvinyl acetate-polyethylene glycol graft copolymer precipitation inhibitor is SOLUPLUS®; the antioxidant comprises one or more selected from butylated hydroxy anisole (BHA) and butylated hydroxy toluene (BHT); the sweetening agent is present and comprises one or more selected from sucralose and ammonium glycyrrhizinate; and the flavoring agent is present and comprises vanilla.

In some embodiments, an oral liquid pharmaceutical composition of enzalutamide as described herein comprises:
(i) about 3% w/w of enzalutamide;
(ii) about 5% w/w of a flavored oil;
(iii) about 12% w/w of a vegetable oil;
(iv) about 15% w/w of the oleoyl polyoxyl-6-glyceride co-surfactant;
(v) about 17% w/w of the tocopherol polyethylene glycol succinate surfactant;
(vi) about 19% w/w of the diethylene glycol monoethyl ether co-solvent;
(vii) about 0.75% w/w of the polyvinyl caprolactam-polyvinyl acetate-polyethylene glycol graft copolymer precipitation inhibitor;
(viii) about 28% w/w of the polyoxyethylene castor oil surfactant,
(ix) an antioxidant;
(x) optionally, a sweetening agent; and
(xi) optionally, a flavoring agent.

In further specific embodiments of the foregoing, the flavored oil is peppermint oil; the vegetable oil is corn oil; the oleoyl polyoxyl-6-glyceride co-surfactant is LABRAFIL® M 1944 CS; the tocopherol polyethylene glycol succinate surfactant is vitamin E TPGS; the diethylene glycol monoethyl ether co-solvent is TRANSCUTOL® HP; the polyvinyl caprolactam-polyvinyl acetate-polyethylene glycol graft copolymer precipitation inhibitor is SOLUPLUS®; the polyoxyethylene castor oil surfactant is KOLLIPHOR® ELP; the antioxidant comprises one or more selected from butylated hydroxy anisole (BHA) and butylated hydroxy toluene (BHT); the sweetening agent is present and comprises one or more selected from sucralose and ammonium glycyrrhizinate; and the flavoring agent is present and comprises vanilla.

In some embodiments, an oral liquid pharmaceutical composition of enzalutamide as described herein comprises:
(i) about 3% w/w of enzalutamide;
(ii) about 5% w/w of a flavored oil;
(iii) about 10% w/w of a vegetable oil;
(iv) about 7% w/w of the propylene glycol monolaurate Type II co-surfactant;
(v) about 15% w/w of the tocopherol polyethylene glycol succinate surfactant;
(vi) about 60% w/w of the polyethylene glycol caprylic/capric glyceride solubilizer;
(vii) about 0.5% w/w of the polyvinyl caprolactam-polyvinyl acetate-polyethylene glycol graft copolymer precipitation inhibitor;
(viii) an antioxidant;
(ix) optionally, a sweetening agent; and
(x) optionally, a flavoring agent.

In further specific embodiments of the foregoing, the flavored oil is peppermint oil; the vegetable oil is corn oil; the propylene glycol monolaurate Type II co-surfactant is CAPMUL® PG-12; the tocopherol polyethylene glycol succinate surfactant is D-α-tocopherol polyethylene glycol 1000 succinate surfactant; the polyethylene glycol caprylic/capric glycerides solubilizer is ACCONON® MC8-2; the polyvinyl caprolactam-polyvinyl acetate-polyethylene glycol graft copolymer precipitation inhibitor is SOLUPLUS®; the antioxidant comprises one or more selected from butylated hydroxy anisole (BHA) and butylated hydroxy toluene (BHT); the sweetening agent is present and comprises one or more selected from sucralose and ammonium glycyrrhizinate; and the flavoring agent is present and comprises vanilla.

### Therapeutic Methods\Uses

The present disclosure also provides uses of the compositions described herein in treatment methods comprising orally administering a liquid oral pharmaceutical enzalutamide composition as described herein to a subject in need thereof. For example, the subject may be a human subject suffering from prostate cancer, and typically is a male subject. The prostate cancer may be advanced prostate cancer, including prostate cancer that does not response to hormone therapy or surgical treatment to lower testosterone, or that has spread to other parts of the body. The composition may be administered at any therapeutically effect dose by any suitable dosing regimen. For example, the composition may be administered once daily in an amount that provides a daily dose of enzalutamide of about 160 mg.

### EXAMPLES

The following specific examples are included as illustrative examples of the compositions described herein. These examples are in no way intended to limit the scope of the disclosure. Other aspects of the disclosure will be apparent to those skilled in the art to which the disclosure pertains.

### Example 1

Formulations containing 160 mg of enzalutamide per unit dose of formulation were prepared with the components indicated in below Table 1, Table 2, Table 3, and Table 4.

TABLE 1

| | | Formulation | Formulation | Formulation | Formulation | Formulation | Formulation |
|---|---|---|---|---|---|---|---|
| | | 1 | 2 | 3 | 4 | 5 | 6 |
| Materials | Category | % | % | % | % | % | % |
| Enzalutamide | API | 3.12% | 4.17% | 3.11% | 2.98% | 2.98% | 2.98% |
| | | (160.00 mg) | (160.00 mg) | (160.00 mg) | (160.00 mg) | (160.00 mg) | (160.00 mg) |
| Peppermint oil | Flavored Oil | 0.98% | 1.30% | 4.86% | 4.65% | 4.65% | 4.65% |
| Refined corn oil | Oil | 3.55% | 4.75% | 11.76% | 11.25% | 11.25% | 11.25% |
| Labrafil ® M | Co- | 12.77% | 17.08% | 12.74% | — | — | — |
| 1944 CS (Oleoyl | Surfactant | | | | | | |
| Polyoxyl-6- | | | | | | | |
| glycerides) | | | | | | | |
| Capmul MCM | Co- | — | — | — | 7.25% | — | — |
| (Glycerol | Surfactant | | | | | | |
| monocaprylocaprate) | | | | | | | |
| Capmul PG 8 | Co- | — | — | — | — | 7.25% | — |
| (Propylene Glycol | Surfactant | | | | | | |
| Monocaprylate) | | | | | | | |
| Capmul PG 12 | Co- | — | — | — | — | — | 7.25% |
| (Propylene Glycol | Surfactant | | | | | | |
| Monolaurate) | | | | | | | |
| Kolliphor ® ELP | Surfactant | 20.48% | — | 29.17% | — | — | — |
| (Polyoxyl 35 | | | | | | | |
| Castor oil) | | | | | | | |
| Vitamin E TPGS | Surfactant | 12.29% | 6.52% | 17.50% | 16.73% | 16.73% | 16.73% |
| (D-α-Tocopherol | | | | | | | |
| Polyethylene | | | | | | | |
| Glycol 1000 | | | | | | | |
| Succinate) | | | | | | | |
| Acconon ® MC8-2 | Solubilizer | 26.19% | 39.11% | — | 55.78% | 55.78% | 55.78% |
| (Polyethylene | | | | | | | |
| glycol | | | | | | | |
| caprylocaproyl | | | | | | | |
| polyoxyl-8 | | | | | | | |
| glycerides) | | | | | | | |
| Transcutol ® HP | Co-Solvent | 19.50% | 26.08% | 19.44% | — | — | — |
| (Diethylene | | | | | | | |
| glycol | | | | | | | |
| monoethyl ether) | | | | | | | |
| BHA (Butylated | Antioxidant | 0.05% | 0.07% | 0.05% | 0.05% | 0.05% | 0.05% |
| Hydroxy Anisole) | | | | | | | |
| BHT (Butylated | Antioxidant | 0.05% | 0.07% | 0.05% | 0.05% | 0.05% | 0.05% |
| Hydroxy Toluene) | | | | | | | |
| Soluplus ® | Precipitation | 0.78% | 0.52% | 0.78% | 0.74% | 0.74% | 0.74% |
| (Polyvinyl | inhibitor | | | | | | |
| caprolactam- | | | | | | | |
| polyvinyl | | | | | | | |
| acetate- | | | | | | | |
| polyethylene | | | | | | | |
| glycol-co- | | | | | | | |
| polymer) | | | | | | | |
| Sucralose | Sweetening | 0.20% | 0.26% | 0.49% | 0.46% | 0.46% | 0.46% |
| | Agent | | | | | | |
| Vanilla | Flavoring | 0.01% | 0.01% | 0.01% | 0.01% | 0.01% | 0.01% |
| | Agent | | | | | | |
| Ammonium | Sweetening | 0.05% | 0.07% | 0.05% | 0.05% | 0.05% | 0.05% |
| Glycyrrhizinate | Agent | | | | | | |

| | | | | | | |
|---|---|---|---|---|---|---|
| Total | 100% | 100% | 100% | 100% | 100% | 100% |

TABLE 2

| | | For- | For- | For- |
|---|---|---|---|---|
| | | mulation | mulation | mulation |
| | | 7 | 8 | 9 |
| Materials | Category | % | % | % |
| Enzalutamide | API | 3.03% | 2.73% | 3.11% |
| | | (160.00 | (160.00 | (160.00 |
| | | mg) | mg) | mg) |
| Peppermint oil | Flavored Oil | 4.73% | 4.26% | 0.97% |
| Refined corn oil | Oil | 11.46% | 10.32% | 3.54% |
| Labrafil ® M 1944 | Co- | 15.00% | — | 12.74% |
| CS | Surfactant | | | |
| (Oleoyl Polyoxyl- | | | | |
| 6-glycerides) | | | | |
| Capmul PG 12 | Co- | — | 6.65% | — |
| (Propylene Glycol | Surfactant | | | |
| Monolaurate) | | | | |
| Kolliphor ® ELP | Surfactant | 28.41% | — | 20.42% |
| (Polyoxyl 35 | | | | |
| Castor oil) | | | | |
| Vitamin E TPGS | Surfactant | 17.05% | 15.35% | 12.25% |
| (D-α-Tocopherol | | | | |
| Polyethylene | | | | |
| Glycol 1000 | | | | |
| Succinate) | | | | |
| Acconon ® MC8-2 | Solubilizer | — | 59.70% | 26.11% |
| (Polyethylene | | | | |
| glycol | | | | |
| capry locaproyl | | | | |
| polyoxyl-8 | | | | |
| glycerides) | | | | |
| Transcutol ® HP | Co-Solvent | 18.94% | — | 19.50% |
| (Diethylene glycol | | | | |
| monoethyl ether) | | | | |
| BHA (Butylated | Antioxidant | 0.05% | 0.04% | 0.05% |
| Hydroxy Anisole) | | | | |
| BHT (Butylated | Antioxidant | 0.05% | 0.04% | 0.05% |
| Hydroxy Toluene) | | | | |
| Soluplus ® | Precipitation | 0.76% | 0.68% | 0.78% |
| (Polyvinyl | inhibitor | | | |
| caprolactam- | | | | |
| polyvinyl acetate- | | | | |
| polyethylene | | | | |
| glycol-co-polymer) | | | | |
| Sucralose | Sweetening | 0.47% | 0.17% | 0.49% |
| | Agent | | | |
| Vanilla | Flavoring | 0.01% | 0.01% | 0.01% |
| | Agent | | | |
| Ammonium | Sweetening | 0.05% | 0.04% | 0.05% |
| Glycyrrhizinate | Agent | | | |

| | | | |
|---|---|---|---|
| Total | 100% | 100% | 100% |

TABLE 3

| | | Formulation | Formulation | Formulation | Formulation | Formulation |
|---|---|---|---|---|---|---|
| | | 10 | 11 | 12 | 13 | 14 |
| Materials | Category | % | % | % | % | % |
| Enzalutamide | API | 2.63% | 2.55% | 2.55% | 2.57% | 2.95% |
| | | (160.00 mg) | (160.00 mg) | (160.00 mg) | (160.00 mg) | (160.00 mg) |
| Peppermint oil | Flavored Oil | 4.11% | 3.99% | 3.99% | 4.01% | — |
| Refined corn oil | Oil | 9.95% | 9.65% | 9.65% | 9.71% | — |
| Capmul PG 12 | Co- | 6.42% | 6.22% | 6.22% | 6.26% | 7.20% |
| (Propylene Glycol | Surfactant | | | | | |
| Monolaurate) | | | | | | |
| Vitamin E TPGS | Surfactant | 14.81% | 14.35% | 14.35% | 14.45% | 16.62% |
| (D-α-Tocopherol | | | | | | |
| Polyethylene | | | | | | |
| Glycol 1000 | | | | | | |
| Succinate) | | | | | | |
| Acconon ® MC8-2 | Solubilizer | 57.58% | 55.82% | 57.42% | 56.18% | 64.64% |
| (Polyethylene | | | | | | |
| glycol | | | | | | |
| caprylocaproyl | | | | | | |
| polyoxyl-8 | | | | | | |
| glycerides) | | | | | | |
| Soluplus ® | Precipitation | 0.66% | 0.64% | 0.64% | — | 0.74% |
| (Polyvinyl | inhibitor | | | | | |
| caprolactam- | | | | | | |
| polyvinyl acetate- | | | | | | |
| polyethylene | | | | | | |
| glycol-co- | | | | | | |
| polymer) | | | | | | |
| Purified water | Solvent | 1.65% | 3.19% | 1.59% | 3.21% | 3.69% |
| DL Methionine | Antioxidant | 0.05% | 0.05% | 0.05% | 0.05% | 0.06% |
| L-Arginine | Antioxidant | — | — | — | — | — |
| L-Cysteine | Antioxidant | — | — | — | — | — |
| Sucralose | Sweetening | 1.32% | 1.28% | 1.28% | 1.28% | 1.48% |
| | Agent | | | | | |
| Vanilla 501441 A | Flavoring | 0.20% | 0.19% | 0.19% | 0.19% | 0.22% |
| | Agent | | | | | |
| Ammonium | Sweetening | 0.16% | 1.88% | 1.88% | 1.89% | 2.18% |
| Glycyrrhizinate | Agent | | | | | |
| Banana Flavor | Flavoring | 0.19% | 0.19% | 0.19% | 0.19% | 0.22% |
| 0527068 | Agent | | | | | |
| Masking Flavor* | Sweetening | 0.02% | — | — | — | — |
| 5600208 | Agent | | | | | |
| BHA (Butylated | Antioxidant | 0.04% | — | — | — | — |
| Hydroxy Anisole) | | | | | | |
| BHT (Butylated | Antioxidant | 0.04% | — | — | — | — |
| Hydroxy Toluene) | | | | | | |

| | | | | | |
|---|---|---|---|---|---|
| Total | 100% | 100% | 100% | 100% | 100% |

TABLE 4

| | | For- | For- | For- |
|---|---|---|---|---|
| | | mulation | mulation | mulation |
| | | 15 | 16 | 17 |
| Materials | Category | % | % | % |
| Enzalutamide | API | 2.55% | 2.55% | 2.63% |
| | | (160.00 | (160.00 | (160.00 |
| | | mg) | mg) | mg) |
| Peppermint oil | Flavored Oil | 3.99% | 3.99% | 4.11% |
| Refined corn oil | Oil | 9.65% | 9.65% | 9.94% |
| Capmul PG 12 | Co- | 6.22% | 6.22% | 6.40% |
| (Propylene Glycol | Surfactant | | | |
| Monolaurate) | | | | |
| Vitamin E TPGS | Surfactant | 14.35% | 14.35% | 14.78% |
| (D-α-Tocopherol | | | | |
| Polyethylene | | | | |
| Glycol 1000 | | | | |
| Succinate) | | | | |
| Acconon ® MC8-2 | Solubilizer | 55.82% | 55.82% | 57.48% |
| (Polyethylene glycol | | | | |
| caprylocaproyl | | | | |
| polyoxyl-8 | | | | |
| glycerides) | | | | |
| Soluplus ® (Polyvinyl | Precipitation | 0.64% | 0.64% | 0.66% |
| caprolactam-polyvinyl | inhibitor | | | |
| acetate-polyethylene | | | | |
| glycol-co-polymer) | | | | |
| Purified water | Solvent | 3.19% | 3.19% | 1.64% |
| DL Methionine | Antioxidant | — | — | 0.05% |
| L-Arginine | Antioxidant | 0.05% | — | — |
| L-Cysteine | Antioxidant | -- | 0.05% | |
| Sucralose | Sweetening | 1.28% | 1.28% | 1.31% |
| | Agent | | | |
| Vanilla 501441 A | Flavoring | 0.19% | 0.19% | 0.20% |
| | Agent | | | |
| Ammonium | Sweetening | 1.88% | 1.88% | 0.41% |
| Glycyrrhizinate | Agent | | | |
| Banana Flavor | Flavoring | 0.19% | 0.19% | 0.20% |
| 0527068 | Agent | | | |
| Masking Flavor | Sweetening | — | — | 0.20% |
| 5600208* | Agent | | | |
| BHA | Antioxidant | — | — | — |
| (Buty lated Hydroxy | | | | |
| Anisole) | | | | |
| BHT | Antioxidant | — | — | — |
| (Buty lated Hydroxy | | | | |
| Toluene) | | | | |
| Total | | 100% | 100% | 100% |

*Masking Flavor was supplied by Bell Flavors & Fragrances GmbH and is chemically similar to ammonium glycyrrhizinate.

In vitro dissolution and in vivo pharmacokinetic properties of the formulations were assessed using four XTANDI® capsules per dissolution vessel as a reference product. As noted above, XTANDI® capsules are soft gelatin capsules containing 40 mg of enzalutamide in a liquid formulation.

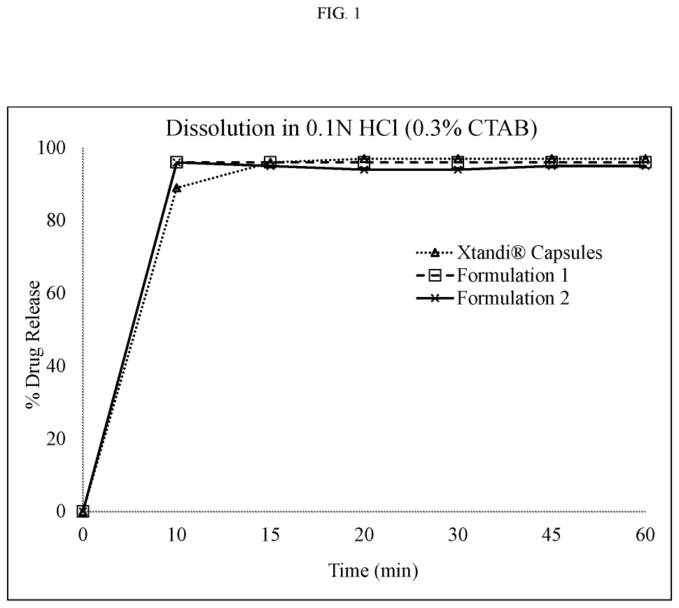

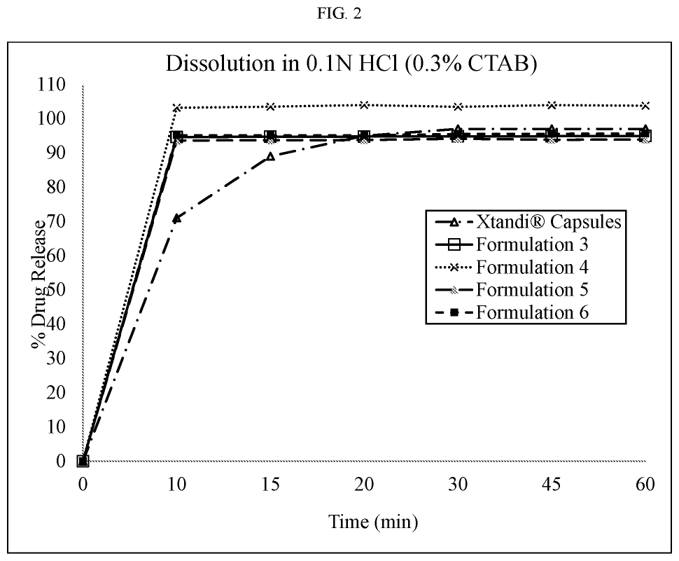

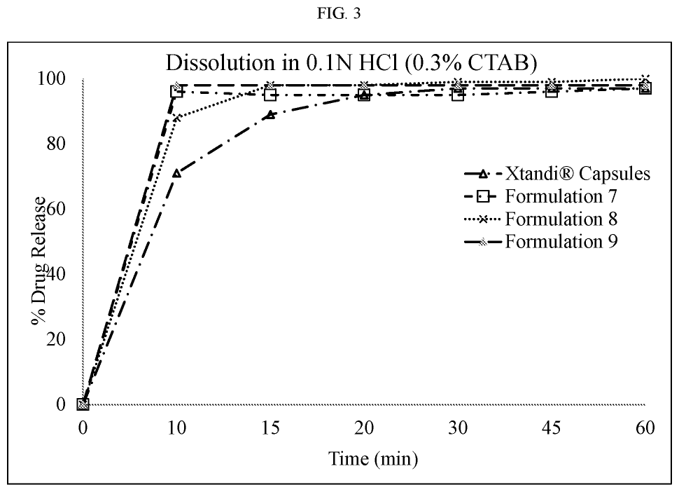

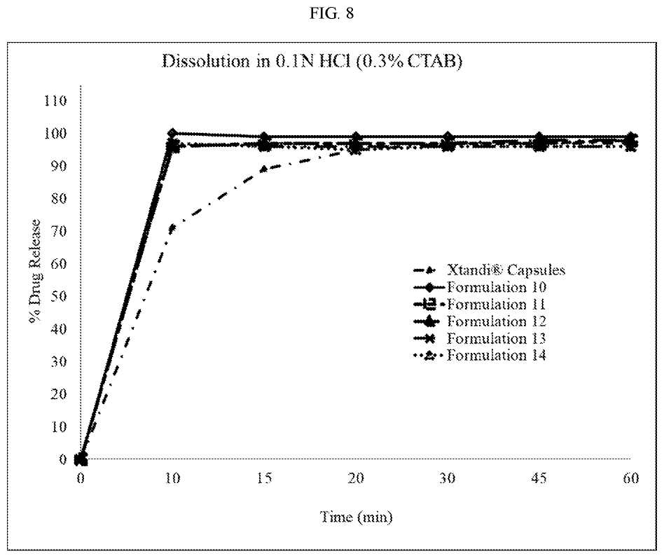

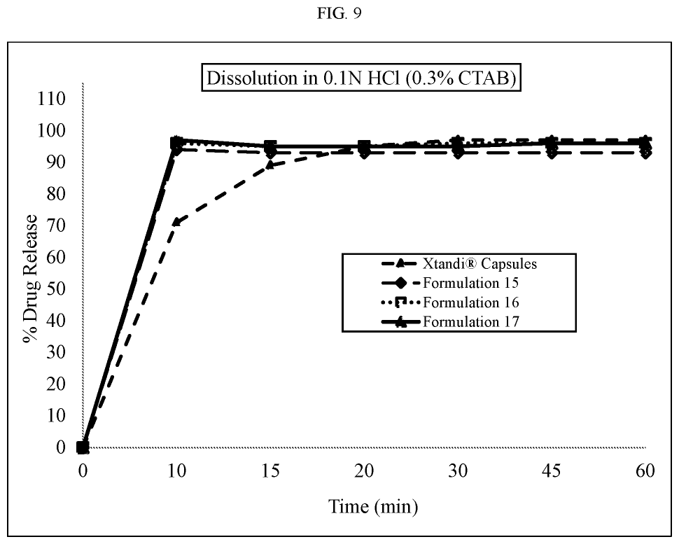

In vitro dissolution testing was performed in accordance with USP Type II, paddle at 50 rpm in 900 mL of 0.1N HCl with 0.3% cetyl trimethyl ammonium bromide (CTAB) dissolution medium, maintained at 37° C.±0.5° C. The release profiles of Formulations 1 and 2 are shown in FIG. 1. The release profiles of Formulations 3, 4, 5 and 6 are shown in FIG. 2. The release profiles of Formulations 7, 8 and 9 are shown in FIG. 3. The release profiles of Formulations 10, 11, 12, 13, and 14 are shown in FIG. 8. The release profiles of Formulations 15, 16, and 17 are shown in FIG. 9. The formulations exhibited complete release of enzalutamide and similar release of enzalutamide as compared to the XTANDI® capsules.

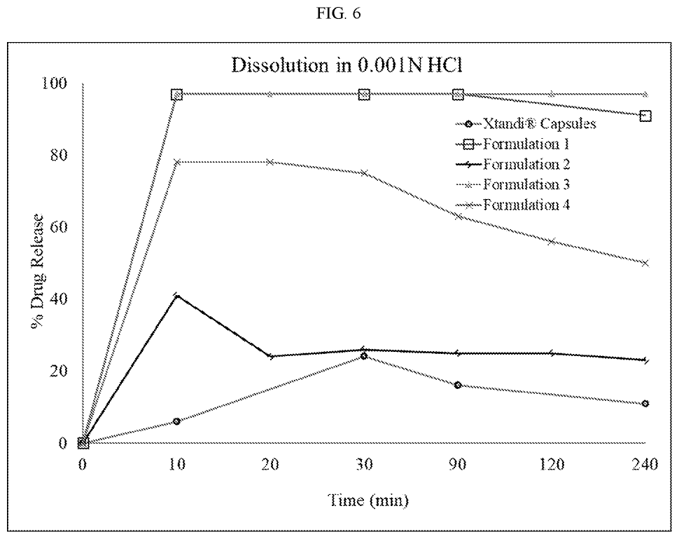

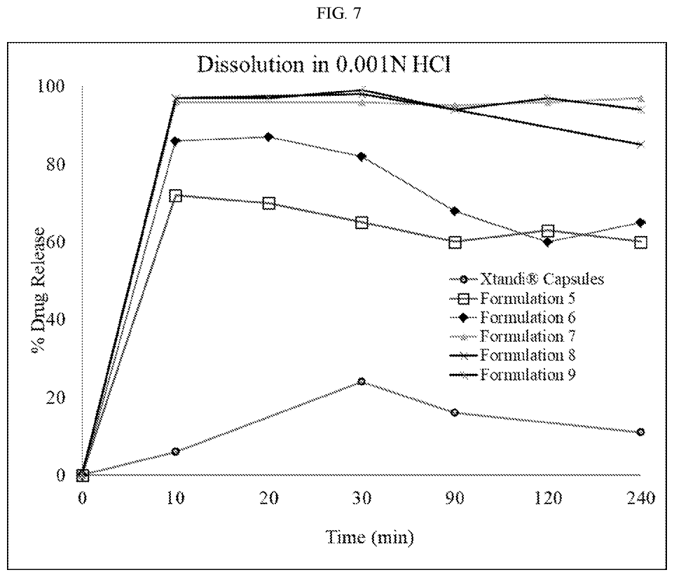

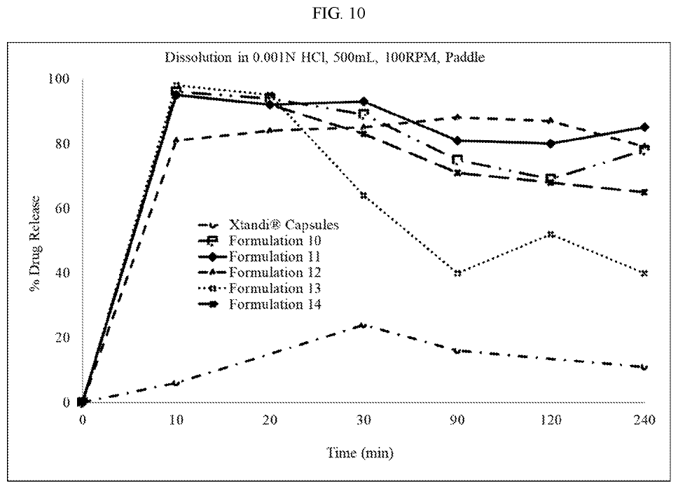

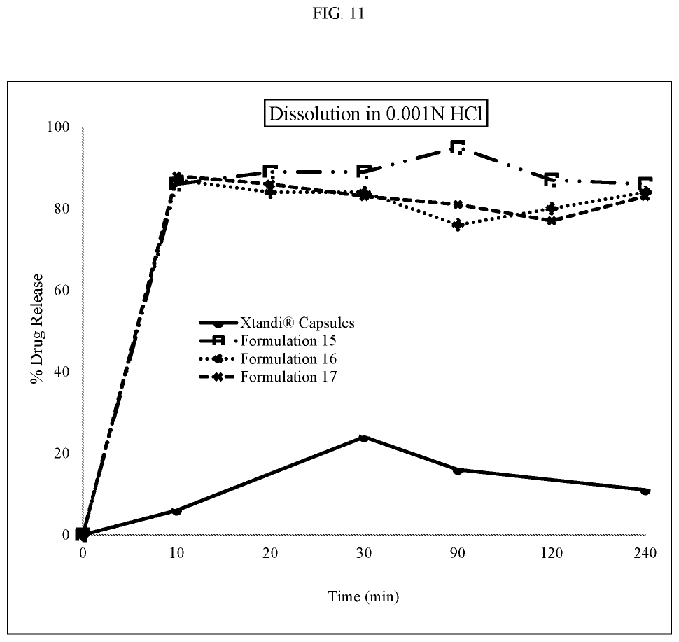

In vitro dissolution testing also was performed in accordance with USP Type II, paddle at 100 rpm in 500 mL of 0.001N HCl dissolution medium (without CTAB), maintained at 37° C.±0.5° C. The release profiles are shown in FIGS. 6 and 7, where the release profiles of Formulations 1, 2, 3 and 4 are shown in FIG. 6, and the release profiles of Formulations 5, 6, 7, 8 and 9 are shown in FIG. 7. The release profiles of Formulations 10, 11, 12, 13, and 14 are shown in FIG. 10. The release profiles of Formulations 15, 16, and 17 are shown in FIG. 11. As reported in the table below and the figures, all Formulations released more than 40% enzalutamide in 10 minutes, as compared to XTANDI® capsules, which released less than 10%.

| Time | Formulations (% release) | | | | | | | | | |
|---|---|---|---|---|---|---|---|---|---|---|
| (mins) | XTANDI ® | 1 | 2 | 3 | 4 | 5 | 6 | 7 | 8 | 9 |
| 10 | 6 | 97 | 41 | 97 | 78 | 72 | 86 | 96 | 97 | 97 |
| 20 | — | — | 24 | 97 | 78 | 70 | 87 | — | — | 97 |
| 30 | 24 | 97 | 26 | 97 | 75 | 65 | 82 | 96 | 98 | 99 |
| 90 | 16 | 97 | 25 | 97 | 63 | 60 | 68 | 95 | 94 | 94 |
| 120 | — | — | 25 | 97 | 56 | 63 | 60 | 96 | — | 97 |
| 240 | 11 | 91 | — | 97 | 50 | 60 | 65 | 97 | 85 | 94 |

| | |
|---|---|
| Time | Formulations (% release) |

| (mins) | 10 | 11 | 12 | 13 | 14 | 15 | 16 | 17 |
|---|---|---|---|---|---|---|---|---|
| 10 | 96 | 95 | 81 | 98 | 95 | 86 | 87 | 88 |
| 20 | 94 | 92 | 84 | 95 | 92 | 89 | 84 | 86 |
| 30 | 89 | 93 | 85 | 64 | 83 | 89 | 84 | 83 |
| 90 | 75 | 81 | 88 | 40 | 71 | 95 | 76 | 81 |
| 120 | 69 | 80 | 87 | 52 | 68 | 87 | 80 | 77 |
| 240 | 78 | 85 | 79 | 48 | 65 | 86 | 84 | 83 |

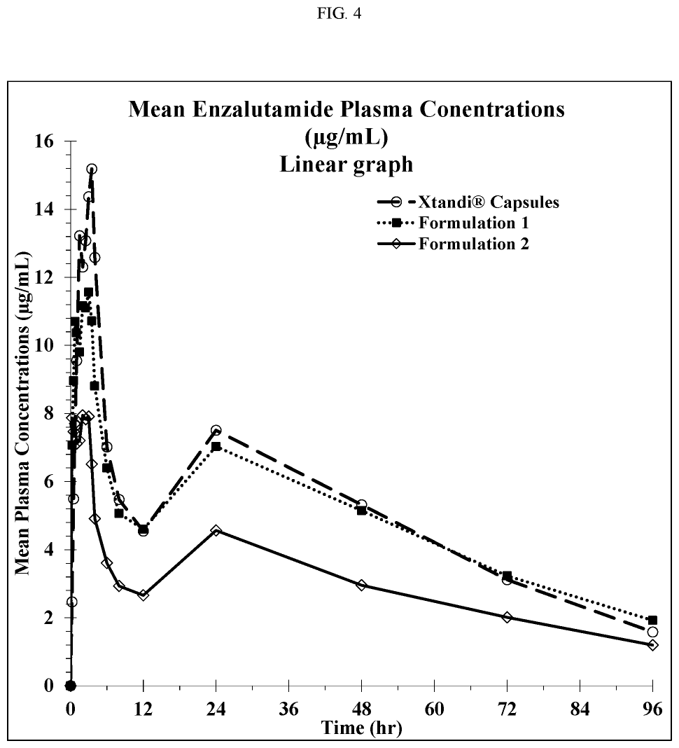

For in vivo testing, 160 mg doses of the enzalutamide Formulation 1 and Formulation 2 as described above were administered to male beagle dogs, and plasma levels of enzalutamide were determined periodically over 96 hours. FIG. 4 shows the mean plasma concentration-time profiles. As shown in the figure, Formulation 1 exhibited a plasma concentration-time profile similar to that of the equivalent dose of XTANDI® capsules (four 40 mg capsules), while Formulation 2 achieved lower plasma levels, but with a similar curve.

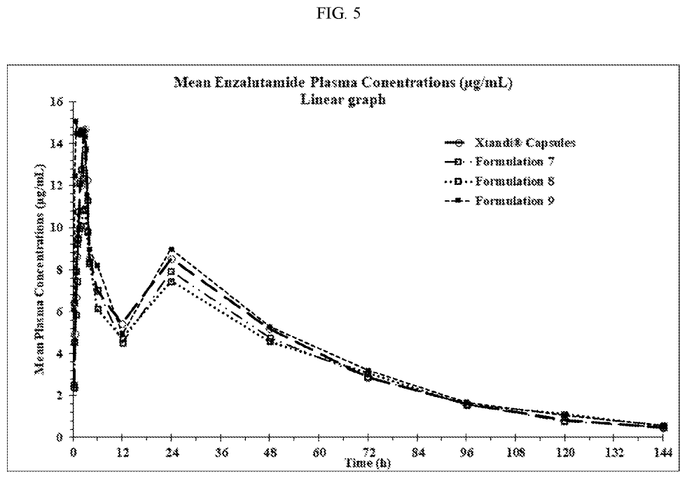

Additionally, 160 mg doses of the enzalutamide Formulations 7, 8 and 9 as described above were administered to male beagle dogs, and plasma levels of enzalutamide were determined periodically over 144 hours. FIG. 5 shows the mean plasma concentration-time profiles. As shown in the figure, Formulations 7, 8 and 9 exhibited a plasma concentration-time profile similar to that of the equivalent dose of XTANDI® capsules (four 40 mg capsules).

### Example 2

The bitterness of Formulations 1, 2, 7, and 9 described above was assessed using the Insent Taste Sensing System TS-5000Z. The TS-5000Z instrument is provided with multiple artificial lipid membranes having different characteristics designed to assess different taste aspects (bitter, salt, sour, astringent). The instrument measures the electric potential of the artificial lipid membrane surface of the instrument, which is affected by test substances contacted to the membrane. For example, test substances may change the electric potential through electrostatic interaction with hydrophilic groups of the lipid membrane, through hydrophobic interaction with hydrophobic groups of the lipid membrane, etc. Thus, measuring changes in potential of the membranes allows the detection of taste aspects, such as bitterness. For these experiments, test samples were prepared as 0.01% solutions of the enzalutamide formulation in 10 mM KCl, and a bitterness standard solution of 0.01 mM Quinine Hydrochloride was used.

As reported in the table below, Formulations 1, 2, 7, 8 and 9 are not bitter as compared to the standard bitterness (Quinine Hydrochloride) solution. Formulation 8 showed some bitterness aftertaste, and Formulations 1, 2 and 9 showed minimal bitterness aftertaste, while Formulation 7 showed no bitterness aftertaste.

| | | Membrane | Membrane |
|---|---|---|---|
| | | Potential | Potential |
| | | for Bitter | for After |
| Sr. No. | Description | Taste (V) | Taste (V) |
| 1 | Quinine HCl | 48.60 | 3.04 |
| 2 | Formulation 1 | 9.58 | 1.23 |
| 3 | Formulation 2 | 10.87 | 1.68 |
| 4 | Formulation 7 | −0.75 | 0.17 |
| 5 | Formulation 8 | 17.02 | 3.39 |
| 6 | Formulation 9 | 8.33 | 1.45 |

### Example 3

The stability of Formulations 8, 10 and 17 as described in Example 1 were assessed at 1 month and 3 months at 40° C. and 75% relative humidity. The results of this study are shown in the below table.

| | | Impurity | | |
|---|---|---|---|---|
| | | % | % Highest | % Total |
| Formulation | | Dioxoimidazoline | individual | Im- |
| (antioxidant) | Stage | impurity | Impurity | purities |
| 08 | Intial | 0.32 | 0.08 | 0.45 |
| (BHA, BHT) | 1M, 40° C./75% | 0.44 | BLOQ | 0.49 |
| | RH | | | |
| | 3M, 40° C./75% | 0.53 | 0.06 | 0.59 |
| | RH | | | |
| 10 | Intial | BLOQ | 0.05 | 0.05 |
| BHA, BHT, | 1M, 40° C./75% | BLOQ | BLOQ | BLOQ |
| DL- | RH | | | |
| Methionine) | 3M, 40° C./75% | BLOQ | BLOQ | BLOQ |
| | RH | | | |
| 17 | Intial | BLOQ | BLOQ | BLOQ |
| (DL- | 1M, 40° C./75% | BLOQ | BLOQ | BLOQ |
| Methionine) | RH | | | |
| | 3M, 40° C./75% | BLOQ | BLOQ | BLOQ |
| | RH | | | |

BLOQ = Below limit of quantification, which was 0.11% w/w for Dioxoimidazoline Impurity, 0.05% w/w for Highest Individual Impurity, and 0.11 % for Total Impurities.

The target impurity limits for each category were: Dioxoimidazoline Impurity-0.2% w/w; Highest Individual Impurity-0.2% w/w; and Total Impurities-1.0% w/w. The formulations prepared with DL-methionine as the antioxidant (with or without BHA and BHT) satisfied these targets. The results showed that Formulations 10 and 17 were stable at three months, satisfying the guidelines of the International Council for Harmonisation of Technical Requirements for Pharmaceuticals for Human Use (ICH) limits on impurities.

### Example 4

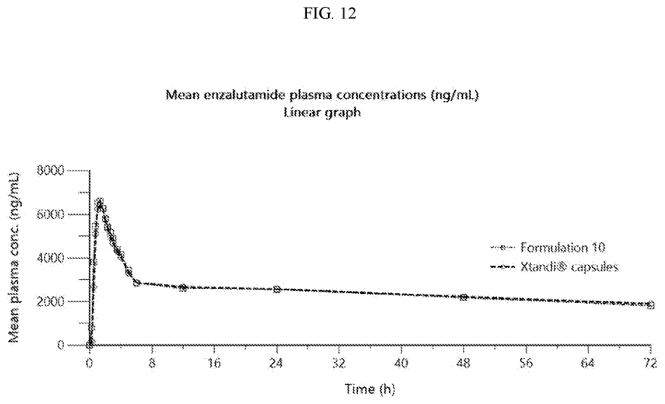

A two-treatment, two-sequence, randomized, crossover study was performed to compare the bioavailability of Formulation 10 as described in Table 3 at dose of 160 mg versus XTANDI® (enzalutamide) capsules (4×400 mg) in 18 healthy adult male humans under fasting conditions. In each study period, a single dose of enzalutamide composition was administered orally in the morning following a 10-hour overnight fast. Fasting condition was maintained up to 4 hours following dosing. Enzalutamide plasma concentrations were quantified through serial blood sampling and pharmacokinetic (PK) parameters determined by non-compartmental analysis. FIG. 12 reports the mean plasma concentration-time profiles. Peak plasma concentration (Cₘₐₓ), area under the plasma concentration-time curve out to 72 hours (AUC_{0-72 h}), and time to peak plasma concentration (Tₘₐₓ) are shown in the table below. AUC values were calculated through a linear-up and log-down trapezoidal method. The results showed that Formulation 10 was comparable to XTANDI®.

What is claimed is:

1. An oral liquid pharmaceutical composition of enzalutamide, comprising:
(i) about 2.5% to about 6% w/w of enzalutamide;
(ii) optionally, about 0.5% to about 25% w/w of an oil selected from one or more of a vegetable oil, a flavored oil, and an essential oil;
(iii) about 5% to about 85% w/w of one or both of a surfactant and a solubilizer, wherein the surfactant comprises one or more selected from tocopherol polyethylene glycol succinate surfactants, polyoxyethylene castor oil surfactants, polyoxyl hydrogenated castor oil surfactants, polyoxyl hydroxy stearate surfactants, lauroyl polyoxylglyceride and lauroyl macrogoglyceride surfactants, stearoyl polyoxylglyceride and stearoyl macrogoglyceride surfactants, and polyoxyl stearate surfactants, and the solubilizer comprises one or more selected from polyethylene glycol caprylic/capric glyceride solubilizers, a polyglyceryl oleate solubilizers, and polyoxyethylene sorbitan monooleate solubilizers;
(iv) about 5% to about 25% w/w of a co-surfactant selected from one or more of propylene glycol monolaurate Type II surfactants, oleoyl polyoxyl-6-glyceride surfactants, linoleoyl macrogolglyceride and linoleoyl polyoxylglyceride surfactants, lauroyl macrogolglyceride and lauroyl polyoxylglyceride surfactants, glycerol monocaprylocaprate Type I surfactants, and propylene glycol monocaprylate Type II surfactants;
(v) about 0.01% to about 1% w/w of an antioxidant comprising one or more selected from DL-methionine, butylated hydroxy anisole (BHA), butylated hydroxy toluene (BHT), arginine, cysteine, ascorbic palmitate, sodium metabisulfite, sodium thiosulfate, propyl gallate, gamma linolenic acid, ethylenediaminetetraacetic acid (EDTA), ascorbic acid, and alpha-tocopherol;
(vi) optionally, about 0.5% to about 5% w/w of precipitation inhibitor selected one or more of polyvinyl caprolactam-polyvinyl acetate-polyethylene glycol graft copolymer solubilizers, polyoxyethylene polyoxypropylene glycol solubilizers, polyvinylpyrrolidone solubilizers, vinylpyrrolidone-vinyl acetate copolymer solubilizers, polyvinyl alcohol/polyethylene glycol graft copolymer solubilizers, hydroxypropyl methylcellulose solubilizers, and hypromellose acetate succinate solubilizers; and
(vii) optionally, about 1% to about 5% w/w water.

2. An oral liquid pharmaceutical composition of enzalutamide, comprising:
(i) about 2.5% to about 6% w/w of enzalutamide;
(ii) optionally, about 0.5% to about 25% w/w of an oil selected from one or more of a vegetable oil, a flavored oil, and an essential oil;
(iii) about 5% to about 65% w/w of a surfactant, wherein the surfactant comprises one or more selected from tocopherol polyethylene glycol succinate surfactants, polyoxyethylene castor oil surfactants, polyoxyl hydrogenated castor oil surfactants, polyoxyl hydroxy stearate surfactants, lauroyl polyoxylglyceride and lauroyl macrogoglyceride surfactants, stearoyl polyoxylglyceride and stearoyl macrogoglyceride surfactants, and polyoxyl stearate surfactants;
(iv) about 5% to about 30% w/w of a co-surfactant selected from one or more of propylene glycol monolaurate Type II surfactants, oleoyl polyoxyl-6-glyceride surfactants, linoleoyl macrogolglyceride and linoleoyl polyoxylglyceride surfactants, lauroyl macrogolglyceride and lauroyl polyoxylglyceride surfactants, glycerol monocaprylocaprate Type I surfactants, and propylene glycol monocaprylate Type II surfactants;
(v) about 0.01% to about 1% w/w of an antioxidant comprising one or more selected from DL-methionine, butylated hydroxy anisole (BHA), butylated hydroxy toluene (BHT), arginine, cysteine, ascorbic palmitate, sodium metabisulfite, sodium thiosulfate, propyl gallate, gamma linolenic acid, ethylenediaminetetraacetic acid (EDTA), ascorbic acid, and alpha-tocopherol;
(vi) optionally, about 0.5% to about 5% w/w of precipitation inhibitor selected one or more of polyvinyl caprolactam-polyvinyl acetate-polyethylene glycol graft copolymer solubilizers, polyoxyethylene polyoxypropylene glycol solubilizers, polyvinylpyrrolidone solubilizers, vinylpyrrolidone-vinyl acetate copolymer solubilizers, polyvinyl alcohol/polyethylene glycol graft copolymer solubilizers, hydroxypropyl methylcellulose solubilizers, and hypromellose acetate succinate solubilizers; and
(vii) optionally, about 1% to about 5% w/w water.

3. The composition of claim 1 or claim 2, comprising about 20 to about 45 mg of enzalutamide per 1 mL of the composition.

4. The composition of claim 1 or claim 2, comprising about 160 mg of enzalutamide per 3.5 to 6.0 mL of the composition.

5. The composition of claim 1 or claim 2, comprising about 160 mg of enzalutamide per 6.0 mL of the composition.

6. The composition of any one of the preceding claims, wherein the oil is present.

7. The composition of any one of the preceding claims, wherein the precipitation inhibitor is present.

8. The composition of any one of the preceding claims, wherein the oil and the precipitation inhibitor are present.

9. The composition of any one of the preceding claims, comprising about 5% to about 50% w/w of the surfactant, about 5% to about 20% w/w of the co-surfactant, and water.

10. The composition of any one of the preceding claims, comprising: (a) about 0.5% to about 20% w/w of the oil, optionally comprising about 0.5% to about 5% w/w of the flavored oil or essential oil and about 3% to about 15% w/w of the vegetable oil, (b) about 5% to about 50% w/w of the surfactant, (c) about 5% to about 20% w/w of the co-surfactant, and (d) about 0.5% to about 1% w/w of the precipitation inhibitor.

11. The composition of any one of the preceding claims, wherein the surfactant comprises a tocopherol polyethylene glycol succinate surfactant and a polyoxyethylene castor oil surfactant, optionally wherein the tocopherol polyethylene glycol succinate surfactant is vitamin E TPGS and the polyoxyethylene castor oil surfactant is PEG 35 castor oil.

12. The composition of any one of the preceding claims, wherein the co-surfactant comprises a propylene glycol monolaurate Type II surfactant, an oleoyl polyoxyl-6-glyceride surfactant, glycerol monocaprylocaprate Type I surfactant, or a propylene glycol monocaprylate Type II surfactant, optionally wherein the propylene glycol monolaurate Type II surfactant is CAPMUL® PG-12, the oleoyl polyoxyl-6-glyceride surfactant is LABRAFIL® M 1944 CS, the glycerol monocaprylocaprate Type I surfactant is CAPMUL® MCM, and the propylene glycol monocaprylate Type II surfactant is CAPMUL® PG-8

13. The composition of any one of the preceding claims, wherein the precipitation inhibitor comprises a polyvinyl caprolactam-polyvinyl acetate-polyethylene glycol graft copolymer solubilizer, optionally wherein the precipitation inhibitor is SOLUPLUS®.

14. The composition of any one of the preceding claims, further comprising about 10% to about 35% w/w of a co-solvent, wherein the co-solvent comprises one or more selected from diethylene glycol monoethyl ether, glycofurol, and N-methyl pyrrolidone.

15. The composition of claim 14, wherein the co-solvent is a diethylene glycol monoethyl ether co-solvent.

16. The composition of any one of the preceding claims, comprising or further comprising about 10% to about 65% w/w of a solubilizer, wherein the solubilizer comprises one or more selected from polyethylene glycol caprylic/capric glyceride solubilizers, a polyglyceryl oleate solubilizers, and polyoxyethylene sorbitan monooleate solubilizers.

17. The composition of claim 16, wherein the solubilizer comprises a polyethylene glycol caprylic/capric glycerides solubilizer, optionally wherein the solubilizer is ACCONON® MC8-2.

18. The composition of any one of the preceding claims, wherein the antioxidant comprises one or more selected from DL-methionine, butylated hydroxy anisole (BHA), and butylated hydroxy toluene (BHT).

19. The composition of claim 18, wherein the antioxidant comprises one or more selected from DL-methionine, arginine, and cysteine.

20. The composition of claim 18, wherein the antioxidant comprises DL-methionine.

21. The composition of any one of the preceding claims, further comprising one or more of a sweetening agent and a flavoring agent.

22. The composition of claim 21, wherein the sweetening agent comprises one or more selected from sucralose, ammonium glycyrrhizinate, saccharin, aspartame, acesulfame potassium, cyclamate, erythritol, and neotame and the flavoring agent comprises one or more selected from vanilla flavor, banana flavor, grapefruit flavor, strawberry flavor, raspberry flavor, bubble gum flavor, and caramel flavor.

23. The composition of claim 21, wherein the sweetening agent comprises one or more selected from sucralose and ammonium glycyrrhizinate, and the flavoring agent comprises one or more selected from vanilla flavor and banana flavor.

24. The composition of any one of the preceding claims, wherein the composition comprises a flavored oil selected from one or more of peppermint oil, liquorice oil, butterscotch oil, and aniseed oil.

25. The composition of any one of the preceding claims, wherein the composition comprises a vegetable oil selected from one or more of corn oil, linseed oil, and rapeseed oil.

26. The composition of any one of the preceding claims, wherein:
the oil is present and comprises peppermint oil as a flavored oil and corn oil as a vegetable oil;
the surfactant is a tocopherol polyethylene glycol succinate surfactant or a combination of a tocopherol polyethylene glycol succinate surfactant and a polyoxyethylene castor oil surfactant, optionally, wherein the surfactant is vitamin E TPGS or wherein the surfactant is vitamin E TPGS and PEG 35 castor oil;
the co-surfactant is a propylene glycol monolaurate Type II co-surfactant, an oleoyl polyoxyl-6-glyceride co-surfactant, a glycerol monocaprylocaprate Type I co-surfactant, or a propylene glycol monocaprylate Type II co-surfactant; and
the precipitation inhibitor is present and is a polyvinyl caprolactam-polyvinyl acetate-polyethylene glycol graft copolymer precipitation inhibitor.

27. The composition of any one claims 1-8, comprising:
(i) about 2.5% w/w of enzalutamide;
(ii) about 5% w/w of a flavored oil;
(iii) about 10% w/w of a vegetable oil;
(iv) about 7% w/w of the propylene glycol monolaurate Type II co-surfactant;
(v) about 15% w/w of the tocopherol polyethylene glycol succinate surfactant;
(vi) about 60% w/w of the polyethylene glycol caprylic/capric glyceride solubilizer;
(vii) about 0.5% w/w of the polyvinyl caprolactam-polyvinyl acetate-polyethylene glycol graft copolymer precipitation inhibitor;
(viii) the antioxidant;
(ix) optionally, the sweetening agent;
(x) optionally, the flavoring agent; and
(xi) water is present in an amount of about 1.5% w/w.

28. The composition of claim 27, wherein:
the flavored oil is peppermint oil;
the vegetable oil is corn oil;
the propylene glycol monolaurate Type II co-surfactant is CAPMUL® PG-12;
the tocopherol polyethylene glycol succinate surfactant is D-α-tocopherol polyethylene glycol 1000 succinate surfactant;
the polyethylene glycol caprylic/capric glycerides solubilizer is ACCONON® MC8-2;
the polyvinyl caprolactam-polyvinyl acetate-polyethylene glycol graft copolymer precipitation inhibitor is SOLUPLUS®;
the antioxidant comprises one or more selected from DL methionine, butylated hydroxy anisole (BHA), and butylated hydroxy toluene;
the sweetening agent is present and comprises one or more selected from sucralose and ammonium glycyrrhizinate; and
the flavoring agent is present and comprises vanilla and banana.

29. The composition of any one of claims 1-8, comprising:
(i) about 2.5% w/w of enzalutamide;
(ii) about 5% w/w of a flavored oil;
(iii) about 10% w/w of a vegetable oil;
(iv) about 7% w/w of the propylene glycol monolaurate Type II co-surfactant;
(v) about 15% w/w of the tocopherol polyethylene glycol succinate surfactant;
(vi) about 60% w/w of the polyethylene glycol caprylic/capric glyceride solubilizer;
(vii) about 0.5% w/w of the polyvinyl caprolactam-polyvinyl acetate-polyethylene glycol graft copolymer precipitation inhibitor;
(viii) the antioxidant;
(ix) optionally, the sweetening agent;
(x) optionally, the flavoring agent; and
(xi) water is present in an amount of about 3% w/w.

30. The composition of claim 29, wherein:
the flavored oil is peppermint oil;
the vegetable oil is corn oil;
the propylene glycol monolaurate Type II co-surfactant is CAPMUL® PG-12;
the tocopherol polyethylene glycol succinate surfactant is D-α-tocopherol polyethylene glycol 1000 succinate surfactant;
the polyethylene glycol caprylic/capric glycerides solubilizer is ACCONON® MC8-2;
the polyvinyl caprolactam-polyvinyl acetate-polyethylene glycol graft copolymer precipitation inhibitor is SOLUPLUS®;
the antioxidant comprises DL methionine;
the sweetening agent is present and comprises one or more selected from sucralose and ammonium glycyrrhizinate; and
the flavoring agent is present and comprises vanilla and banana.

31. The composition of any one of claims 1-8, comprising:
(i) about 2.5% w/w of enzalutamide;
(ii) about 5% w/w of a flavored oil;
(iii) about 10% w/w of a vegetable oil;
(iv) about 7% w/w of the propylene glycol monolaurate Type II co-surfactant;
(v) about 15% w/w of the tocopherol polyethylene glycol succinate surfactant;
(vi) about 60% w/w of the polyethylene glycol caprylic/capric glyceride solubilizer;
(vii) about 0.5% w/w of the polyvinyl caprolactam-polyvinyl acetate-polyethylene glycol graft copolymer precipitation inhibitor;
(viii) the antioxidant;
(ix) optionally, the sweetening agent;
(x) optionally, the flavoring agent; and
(xi) water is present in an amount of about 1.5% w/w.

32. The composition of claim 31, wherein:
the flavored oil is peppermint oil;
the vegetable oil is corn oil;
the propylene glycol monolaurate Type II co-surfactant is CAPMUL® PG-12;
the tocopherol polyethylene glycol succinate surfactant is D-α-tocopherol polyethylene glycol 1000 succinate surfactant;
the polyethylene glycol caprylic/capric glycerides solubilizer is ACCONON® MC8-2;
the polyvinyl caprolactam-polyvinyl acetate-polyethylene glycol graft copolymer precipitation inhibitor is SOLUPLUS®;
the antioxidant comprises DL methionine;
the sweetening agent is present and comprises one or more selected from sucralose and ammonium glycyrrhizinate; and
the flavoring agent is present and comprises vanilla and banana.

33. The composition of any one of claims 1-6, comprising:
(i) about 2.5% w/w of enzalutamide;
(ii) about 5% w/w of a flavored oil;
(iii) about 10% w/w of a vegetable oil;
(iv) about 7% w/w of the propylene glycol monolaurate Type II co-surfactant;
(v) about 15% w/w of the tocopherol polyethylene glycol succinate surfactant;
(vi) about 60% w/w of the polyethylene glycol caprylic/capric glyceride solubilizer;
(vii) the antioxidant;
(viii) optionally, the sweetening agent;
(ix) optionally, the flavoring agent; and
(x) water is present in an amount of about 3% w/w.

34. The composition of claim 33, wherein:
the flavored oil is peppermint oil;
the vegetable oil is corn oil;
the propylene glycol monolaurate Type II co-surfactant is CAPMUL® PG-12;
the tocopherol polyethylene glycol succinate surfactant is D-α-tocopherol polyethylene glycol 1000 succinate surfactant;
the polyethylene glycol caprylic/capric glycerides solubilizer is ACCONON® MC8-2;
the antioxidant comprises DL methionine;
the sweetening agent is present and comprises one or more selected from sucralose and ammonium glycyrrhizinate; and
the flavoring agent in present and comprises vanilla and banana.

35. The composition of any one of claims 1-5, comprising:
(i) about 2.5% w/w of enzalutamide;
(ii) about 7% w/w of the propylene glycol monolaurate Type II co-surfactant;
(iii) about 15% w/w of the tocopherol polyethylene glycol succinate surfactant;
(iv) about 60% w/w of the polyethylene glycol caprylic/capric glyceride solubilizer;
(v) about 0.5% w/w of the polyvinyl caprolactam-polyvinyl acetate-polyethylene glycol graft copolymer precipitation inhibitor;
(vi) the antioxidant;
(vii) optionally, the sweetening agent;
(viii) optionally, the flavoring agent; and
(ix) water is present in an amount of about 3% w/w.

36. The composition of claim 35, wherein:
the propylene glycol monolaurate Type II co-surfactant is CAPMUL® PG-12;
the tocopherol polyethylene glycol succinate surfactant is D-α-tocopherol polyethylene glycol 1000 succinate surfactant;
the polyethylene glycol caprylic/capric glycerides solubilizer is ACCONON® MC8-2;
the polyvinyl caprolactam-polyvinyl acetate-polyethylene glycol graft copolymer precipitation inhibitor is SOLUPLUS®;
the antioxidant comprises DL methionine;
the sweetening agent is present and comprises one or more selected from sucralose and ammonium glycyrrhizinate; and
the flavoring agent is present and comprises vanilla and banana.

37. The composition of any one of claims 1-8, comprising:
(i) about 2.5% w/w of enzalutamide;
(ii) about 5% w/w of a flavored oil;
(iii) about 10% w/w of a vegetable oil;
(iv) about 7% w/w of the propylene glycol monolaurate Type II co-surfactant;
(v) about 15% w/w of the tocopherol polyethylene glycol succinate surfactant;
(vi) about 60% w/w of the polyethylene glycol caprylic/capric glyceride solubilizer;
(vii) about 0.5% w/w of the polyvinyl caprolactam-polyvinyl acetate-polyethylene glycol graft copolymer precipitation inhibitor;
(viii) the antioxidant;
(ix) optionally, the sweetening agent;
(x) optionally, the flavoring agent; and
(xi) water is present in an amount of about 3% w/w.

38. The composition of claim 37, wherein:
the flavored oil is peppermint oil;
the vegetable oil is corn oil;
the propylene glycol monolaurate Type II co-surfactant is CAPMUL® PG-12;
the tocopherol polyethylene glycol succinate surfactant is D-α-tocopherol polyethylene glycol 1000 succinate surfactant;
the polyethylene glycol caprylic/capric glycerides solubilizer is ACCONON® MC8-2;
the polyvinyl caprolactam-polyvinyl acetate-polyethylene glycol graft copolymer precipitation inhibitor is SOLUPLUS®;
the antioxidant comprises arginine;
the sweetening agent is present and comprises one or more selected from sucralose and ammonium glycyrrhizinate; and
the flavoring agent is present and comprises vanilla and banana.

39. The composition of claim 37, wherein:
the flavored oil is peppermint oil;
the vegetable oil is corn oil;
the propylene glycol monolaurate Type II co-surfactant is CAPMUL® PG-12;
the tocopherol polyethylene glycol succinate surfactant is D-α-tocopherol polyethylene glycol 1000 succinate surfactant;
the polyethylene glycol caprylic/capric glycerides solubilizer is ACCONON® MC8-2;
the polyvinyl caprolactam-polyvinyl acetate-polyethylene glycol graft copolymer precipitation inhibitor is SOLUPLUS®;
the antioxidant comprises cysteine;
the sweetening agent is present and comprises one or more selected from sucralose and ammonium glycyrrhizinate; and
the flavoring agent is present and comprises vanilla and banana.

40. The composition of any one of claims 1-8, comprising:
(i) about 2.5% w/w of enzalutamide;
(ii) about 5% w/w of a flavored oil;
(iii) about 10% w/w of a vegetable oil;
(iv) about 7% w/w of the propylene glycol monolaurate Type II co-surfactant;
(v) about 15% w/w of the tocopherol polyethylene glycol succinate surfactant;
(vi) about 60% w/w of the polyethylene glycol caprylic/capric glyceride solubilizer;
(vii) about 0.5% w/w of the polyvinyl caprolactam-polyvinyl acetate-polyethylene glycol graft copolymer precipitation inhibitor;
(viii) the antioxidant;
(ix) optionally, the sweetening agent;
(x) optionally, the flavoring agent; and
(xi) water is present in an amount of about 1.5% w/w.

41. The composition of claim 40, wherein:
the flavored oil is peppermint oil;
the vegetable oil is corn oil;
the propylene glycol monolaurate Type II co-surfactant is CAPMUL® PG-12;
the tocopherol polyethylene glycol succinate surfactant is D-α-tocopherol polyethylene glycol 1000 succinate surfactant;
the polyethylene glycol caprylic/capric glycerides solubilizer is ACCONON® MC8-2;
the polyvinyl caprolactam-polyvinyl acetate-polyethylene glycol graft copolymer precipitation inhibitor is SOLUPLUS®;
the comprises DL methionine;
the sweetening agent is present and comprises one or more selected from sucralose and ammonium glycyrrhizinate; and
the flavoring agent is present and comprises vanilla and banana.

42. The composition of any one of claims 1-8, comprising:
(i) about 3% w/w of enzalutamide;
(ii) about 1.0% w/w of a flavored oil;
(iii) about 3.5% w/w of a vegetable oil;
(iv) about 13% w/w of the oleoyl polyoxyl-6-glyceride co-surfactant;
(v) about 12% w/w of the tocopherol polyethylene glycol succinate surfactant;
(vi) about 20% w/w of the diethylene glycol monoethyl ether co-solvent;
(vii) about 26% w/w of the polyethylene glycol caprylic/capric glyceride solubilizer;
(viii) about 0.75% w/w of the polyvinyl caprolactam-polyvinyl acetate-polyethylene glycol graft copolymer precipitation inhibitor;
(ix) about 21% w/w of the polyoxyethylene castor oil surfactant,
(x) the antioxidant;
(xi) optionally, the sweetening agent; and
(xii) optionally, the flavoring agent.

43. The composition of claim 42, wherein:
the flavored oil is peppermint oil;
the vegetable oil is corn oil;
the oleoyl polyoxyl-6-glyceride co-surfactant is LABRAFIL® M 1944 CS;
the tocopherol polyethylene glycol succinate surfactant is vitamin E TPGS;
the diethylene glycol monoethyl ether co-solvent is TRANSCUTOL® HP;
the polyethylene glycol caprylic/capric glyceride solubilizer is ACCONON® MC8-2;
the polyvinyl caprolactam-polyvinyl acetate-polyethylene glycol graft copolymer precipitation inhibitor is SOLUPLUS®;
the polyoxyethylene castor oil surfactant is KOLLIPHOR® ELP;
the antioxidant comprises one or more selected from butylated hydroxy anisole (BHA) and butylated hydroxy toluene (BHT);
the sweetening agent is present and comprises one or more selected from sucralose and ammonium glycyrrhizinate; and
the flavoring agent is present and comprises vanilla.

44. The composition of any one of claims 1-8, comprising:
(i) about 4% w/w of enzalutamide;
(ii) about 1.5% w/w of a flavored oil;
(iii) about 5% w/w of a vegetable oil;
(iv) about 17% w/w of the oleoyl polyoxyl-6-glyceride co-surfactant;
(v) about 6% w/w of the tocopherol polyethylene glycol succinate surfactant;
(vi) about 25% w/w of the diethylene glycol monoethyl ether co-solvent;
(vii) about 40% w/w of the polyethylene glycol caprylic/capric glyceride solubilizer;
(viii) about 0.5% w/w of the polyvinyl caprolactam-polyvinyl acetate-polyethylene glycol graft copolymer precipitation inhibitor;
(ix) the antioxidant;
(x) optionally, the sweetening agent; and
(xi) optionally, the flavoring agent.

45. The composition of claim 44, wherein:
the flavored oil is peppermint oil;
the vegetable oil is corn oil;
the oleoyl polyoxyl-6-glyceride co-surfactant is LABRAFIL® M 1944 CS;
the tocopherol polyethylene glycol succinate surfactant is D-α-tocopherol polyethylene glycol 1000 succinate surfactant;
the diethylene glycol monoethyl ether co-solvent is TRANSCUTOL® HP;
the polyethylene glycol caprylic/capric glycerides solubilizer is ACCONON® MC8-2;
the polyvinyl caprolactam-polyvinyl acetate-polyethylene glycol graft copolymer precipitation inhibitor is SOLUPLUS®;
the antioxidant comprises one or more selected from butylated hydroxy anisole (BHA) and butylated hydroxy toluene (BHT);
the sweetening agent is present and comprises one or more selected from sucralose and ammonium glycyrrhizinate; and
the flavoring agent is present and comprises vanilla.

46. The composition of any one of claims 1-8, comprising:
(i) about 3% w/w of enzalutamide;
(ii) about 5% w/w of a flavored oil;
(iii) about 12% w/w of a vegetable oil;
(iv) about 13% w/w of the oleoyl polyoxyl-6-glyceride co-surfactant;
(v) about 18% w/w of the tocopherol polyethylene glycol succinate surfactant;
(vi) about 20% w/w of the diethylene glycol monoethyl ether co-solvent;
(vii) about 0.75% w/w of the polyvinyl caprolactam-polyvinyl acetate-polyethylene glycol graft copolymer precipitation inhibitor;
(viii) about 30% w/w of the polyoxyethylene castor oil surfactant,
(ix) the antioxidant;
(x) optionally, the sweetening agent; and
(xi) optionally, the flavoring agent.

47. The composition of claim 46, wherein:
the flavored oil is peppermint oil;
the vegetable oil is corn oil;
the oleoyl polyoxyl-6-glyceride co-surfactant is LABRAFIL® M 1944 CS;
the tocopherol polyethylene glycol succinate surfactant is vitamin E TPGS;
the diethylene glycol monoethyl ether co-solvent is TRANSCUTOL® HP;
the polyvinyl caprolactam-polyvinyl acetate-polyethylene glycol graft copolymer precipitation inhibitor is SOLUPLUS®;
the polyoxyethylene castor oil surfactant is KOLLIPHOR® ELP;
the antioxidant comprises one or more selected from butylated hydroxy anisole (BHA) and butylated hydroxy toluene (BHT);
the sweetening agent is present and comprises one or more selected from sucralose and ammonium glycyrrhizinate; and
the flavoring agent is present and comprises vanilla.

48. The composition of any one of claims 1-8, comprising:
(i) about 3% w/w of enzalutamide;
(ii) about 5% w/w of a flavored oil;
(iii) about 11% w/w of a vegetable oil;
(iv) about 7% w/w of the glycerol monocaprylocaprate Type I co-surfactant;
(v) about 17% w/w of the tocopherol polyethylene glycol succinate surfactant;
(vi) about 55% w/w of the polyethylene glycol caprylic/capric glyceride solubilizer;
(vii) about 0.5% w/w of the polyvinyl caprolactam-polyvinyl acetate-polyethylene glycol graft copolymer precipitation inhibitor;
(viii) the antioxidant;
(ix) optionally, the sweetening agent; and
(x) optionally, the flavoring agent.

49. The composition of claim 48, wherein:
the flavored oil is peppermint oil;
the vegetable oil is corn oil;
the glycerol monocaprylocaprate Type I co-surfactant is CAPMUL® MCM;
the tocopherol polyethylene glycol succinate surfactant is D-α-tocopherol polyethylene glycol 1000 succinate surfactant;
the polyethylene glycol caprylic/capric glycerides solubilizer is ACCONON® MC8-2;
the polyvinyl caprolactam-polyvinyl acetate-polyethylene glycol graft copolymer precipitation inhibitor is SOLUPLUS®;
the antioxidant comprises one or more selected from butylated hydroxy anisole (BHA) and butylated hydroxy toluene (BHT);
the sweetening agent is present and comprises one or more selected from sucralose and ammonium glycyrrhizinate; and
the flavoring agent is present and comprises vanilla.

50. The composition of any one of claims 1-8, comprising:
(i) about 3% w/w of enzalutamide;
(ii) about 5% w/w of a flavored oil;
(iii) about 11% w/w of a vegetable oil;
(iv) about 7% w/w of the propylene glycol monocaprylate Type II co-surfactant;
(v) about 17% w/w of the tocopherol polyethylene glycol succinate surfactant;
(vi) about 55% w/w of the polyethylene glycol caprylic/capric glyceride solubilizer;
(vii) about 0.5% w/w of the polyvinyl caprolactam-polyvinyl acetate-polyethylene glycol graft copolymer precipitation inhibitor;
(viii) the antioxidant;
(ix) optionally, the sweetening agent; and
(x) optionally, the flavoring agent.

51. The composition of claim 50, wherein:
the flavored oil is peppermint oil;
the vegetable oil is corn oil;
the propylene glycol monocaprylate Type II co-surfactant is CAPMUL® PG-8;
the tocopherol polyethylene glycol succinate surfactant is D-α-tocopherol polyethylene glycol 1000 succinate surfactant;
the polyethylene glycol caprylic/capric glycerides solubilizer is ACCONON® MC8-2;
the polyvinyl caprolactam-polyvinyl acetate-polyethylene glycol graft copolymer precipitation inhibitor is SOLUPLUS®;
the antioxidant comprises one or more selected from butylated hydroxy anisole (BHA) and butylated hydroxy toluene (BHT);
the sweetening agent is present and comprises one or more selected from sucralose and ammonium glycyrrhizinate; and
the flavoring agent is present and comprises vanilla.

52. The composition of any one of claims 1-8, comprising:
(i) about 3% w/w of enzalutamide;
(ii) about 5% w/w of a flavored oil;
(iii) about 11% w/w of a vegetable oil;
(iv) about 7% w/w of the propylene glycol monolaurate Type II co-surfactant;
(v) about 17% w/w of the tocopherol polyethylene glycol succinate surfactant;
(vi) about 55% w/w of the polyethylene glycol caprylic/capric glyceride solubilizer;
(vii) about 0.5% w/w of the polyvinyl caprolactam-polyvinyl acetate-polyethylene glycol graft copolymer precipitation inhibitor;
(viii) the antioxidant;
(ix) optionally, the sweetening agent; and
(x) optionally, the flavoring agent.

53. The composition of claim 52, wherein:
the flavored oil is peppermint oil;
the vegetable oil is corn oil;
the propylene glycol monolaurate Type II co-surfactant is CAPMUL® PG-12;
the tocopherol polyethylene glycol succinate surfactant is D-α-tocopherol polyethylene glycol 1000 succinate surfactant;
the polyethylene glycol caprylic/capric glycerides solubilizer is ACCONON® MC8-2;
the polyvinyl caprolactam-polyvinyl acetate-polyethylene glycol graft copolymer precipitation inhibitor is SOLUPLUS®;
the antioxidant comprises one or more selected from butylated hydroxy anisole (BHA) and butylated hydroxy toluene (BHT);
the sweetening agent is present and comprises one or more selected from sucralose and ammonium glycyrrhizinate; and
the flavoring agent is present and comprises vanilla.

54. The composition of any one of claims 1-8, comprising:
(i) about 3% w/w of enzalutamide;
(ii) about 5% w/w of a flavored oil;
(iii) about 12% w/w of a vegetable oil;
(iv) about 15% w/w of the oleoyl polyoxyl-6-glyceride co-surfactant;
(v) about 17% w/w of the tocopherol polyethylene glycol succinate surfactant;
(vi) about 19% w/w of the diethylene glycol monoethyl ether co-solvent;
(vii) about 0.75% w/w of the polyvinyl caprolactam-polyvinyl acetate-polyethylene glycol graft copolymer precipitation inhibitor;
(viii) about 28% w/w of the polyoxyethylene castor oil surfactant,
(ix) the antioxidant;
(x) optionally, the sweetening agent; and
(xi) optionally, the flavoring agent.

55. The composition of claim 54, wherein:
the flavored oil is peppermint oil;
the vegetable oil is corn oil;
the oleoyl polyoxyl-6-glyceride co-surfactant is LABRAFIL® M 1944 CS;
the tocopherol polyethylene glycol succinate surfactant is vitamin E TPGS;
the diethylene glycol monoethyl ether co-solvent is TRANSCUTOL® HP;
the polyvinyl caprolactam-polyvinyl acetate-polyethylene glycol graft copolymer precipitation inhibitor is SOLUPLUS®;
the polyoxyethylene castor oil surfactant is KOLLIPHOR® ELP;
the antioxidant comprises one or more selected from butylated hydroxy anisole (BHA) and butylated hydroxy toluene (BHT);
the sweetening agent is present and comprises one or more selected from sucralose and ammonium glycyrrhizinate; and
the flavoring agent is present and comprises vanilla.

56. The composition of any one of claims 1-8, comprising:
(i) about 3% w/w of enzalutamide;
(ii) about 5% w/w of a flavored oil;
(iii) about 10% w/w of a vegetable oil;
(iv) about 7% w/w of the propylene glycol monolaurate Type II co-surfactant;
(v) about 15% w/w of the tocopherol polyethylene glycol succinate surfactant;
(vi) about 60% w/w of the polyethylene glycol caprylic/capric glyceride solubilizer;
(vii) about 0.5% w/w of the polyvinyl caprolactam-polyvinyl acetate-polyethylene glycol graft copolymer precipitation inhibitor;
(viii) the antioxidant;
(ix) optionally, the sweetening agent; and
(x) optionally, the flavoring agent.

57. The composition of claim 56, wherein:
the flavored oil is peppermint oil;
the vegetable oil is corn oil;
the propylene glycol monolaurate Type II co-surfactant is CAPMUL® PG-12;
the tocopherol polyethylene glycol succinate surfactant is D-α-tocopherol polyethylene glycol 1000 succinate surfactant;
the polyethylene glycol caprylic/capric glycerides solubilizer is ACCONON® MC8-2;
the polyvinyl caprolactam-polyvinyl acetate-polyethylene glycol graft copolymer precipitation inhibitor is SOLUPLUS®;
the antioxidant comprises one or more selected from butylated hydroxy anisole (BHA) and butylated hydroxy toluene (BHT);
the sweetening agent is present and comprises one or more selected from sucralose and ammonium glycyrrhizinate; and
the flavoring agent is present and comprises vanilla.

58. An oral liquid pharmaceutical composition of enzalutamide, comprising:
(i) about 160 mg enzalutamide in a volume of about 2.5 to 6.0 mL;
(ii) about 0.5% to about 25% w/w of an oil selected from one or more of a vegetable oil, a flavored oil, and an essential oil;
(iii) about 5% to about 55% w/w of a surfactant, wherein the surfactant comprises one or more selected from tocopherol polyethylene glycol succinate surfactants, polyoxyethylene castor oil surfactants, polyoxyl hydrogenated castor oil surfactants, polyoxyl hydroxy stearate surfactants, lauroyl polyoxylglyceride and lauroyl macrogoglyceride surfactants, stearoyl polyoxylglyceride and stearoyl macrogoglyceride surfactants, and polyoxyl stearate surfactants;
(iv) about 5% to about 25% w/w of a co-surfactant selected from one or more of oleoyl polyoxyl-6-glyceride surfactants, linoleoyl macrogolglyceride and linoleoyl polyoxylglyceride surfactants, lauroyl macrogolglyceride and lauroyl polyoxylglyceride surfactants, glycerol monocaprylocaprate Type I surfactants, propylene glycol monocaprylate Type II surfactants, and propylene glycol monolaurate Type II surfactants; and
(v) about 0.5% to about 5% w/w of precipitation inhibitor selected one or more of polyvinyl caprolactam-polyvinyl acetate-polyethylene glycol graft copolymer solubilizers, polyoxyethylene polyoxypropylene glycol solubilizers, polyvinylpyrrolidone solubilizers, vinylpyrrolidone-vinyl acetate copolymer solubilizers, polyvinyl alcohol/polyethylene glycol graft copolymer solubilizers, hydroxypropyl methylcellulose solubilizers, and hypromellose acetate succinate solubilizers.

59. An oral liquid pharmaceutical composition of enzalutamide, comprising:
(i) about 3% to about 6% w/w of enzalutamide;
(ii) about 0.5% to about 25% w/w of an oil selected from one or more of a vegetable oil, a flavored oil, and an essential oil;
(iii) about 5% to about 55% w/w of a surfactant, wherein the surfactant comprises one or more selected from tocopherol polyethylene glycol succinate surfactants, polyoxyethylene castor oil surfactants, polyoxyl hydrogenated castor oil surfactants, polyoxyl hydroxy stearate surfactants, lauroyl polyoxylglyceride and lauroyl macrogoglyceride surfactants, stearoyl polyoxylglyceride and stearoyl macrogoglyceride surfactants, and polyoxyl stearate surfactants;
(iv) about 5% to about 25% w/w of a co-surfactant selected from one or more of oleoyl polyoxyl-6-glyceride surfactants, linoleoyl macrogolglyceride and linoleoyl polyoxylglyceride surfactants, lauroyl macrogolglyceride and lauroyl polyoxylglyceride surfactants, glycerol monocaprylocaprate Type I surfactants, propylene glycol monocaprylate Type II surfactants, and propylene glycol monolaurate Type II surfactants; and
(v) about 0.5% to about 5% w/w of precipitation inhibitor selected one or more of polyvinyl caprolactam-polyvinyl acetate-polyethylene glycol graft copolymer solubilizers, polyoxyethylene polyoxypropylene glycol solubilizers, polyvinylpyrrolidone solubilizers, vinylpyrrolidone-vinyl acetate copolymer solubilizers, polyvinyl alcohol/polyethylene glycol graft copolymer solubilizers, hydroxypropyl methylcellulose solubilizers, and hypromellose acetate succinate solubilizers.

60. The composition of any one of the preceding claims, wherein the composition is palatable.

61. The composition of any one of the preceding claims, packaged as a unit dose containing 160 mg enzalutamide.

62. The composition of any one of the preceding claims, wherein the composition does not include caprylocaproyl polyoxyl-8 glyceride solubilizers/surfactants, optionally wherein the excluded caprylocaproyl polyoxyl-8 glyceride solubilizer/surfactant is LABRASOL® and/or LABRASOL® ALF, optionally wherein the composition does not include LABRASOL® and does not include LABRASOL® ALF.

63. The composition of any one of the preceding claims, wherein the composition does not include polyethylene glycol, optionally wherein the excluded polyethylene glycol is polyethylene glycol 300 (PEG 300) and/or polyethylene glycol 400 (PEG 400), optionally wherein the composition does not include PEG 300 and does not include PEG 400.

64. The composition of any one of the preceding claims, wherein the composition does not include propylene glycol.

65. The composition of any one of the preceding claims, wherein the composition does not include ethanol.

66. A method of administering enzalutamide to a subject in need thereof, comprising orally administering a composition according to any one of the preceding claims.

67. A method of treating prostate cancer, comprising orally administering a composition according to any one of claims 1-65 to a subject in need thereof.

68. An oral liquid pharmaceutical composition according to any one of claims 1-65, for use in treating prostate cancer.

69. Use of enzalutamide in the preparation of a medicament for treating prostate cancer, wherein the medicament comprises an oral liquid pharmaceutical composition according to any one of claims 1-65.
